(19) United States
(12) Patent Application Publication
(10) Pub. No.: US 2010/0203531 A1
(43) Pub. Date: Aug. 12, 2010

(54) PREDICTING RESPONSIVENESS TO TEMOZOLOMIDE

(76) Inventors: Jann N. Sarkaria, Rochester, MN (US); Gasper J. Kitange, Rochester, MN (US); Brett L. Carlson, Owatonna, MN (US); Mark A. Schroeder, Rochester, MN (US); Ann C.M. Tuma, Mantorville, MN (US); Paul A. Decker, Rochester, MN (US); Wenting Wu, Rochester, MN (US)

Correspondence Address: FISH & RICHARDSON P.C., PO BOX 1022, MINNEAPOLIS, MN, 55440-1022, US

(21) Appl. No.: 12/664,143

(22) PCT Filed: Jun. 11, 2008
(86) PCT No.: PCT/US08/66586

§ 371 Date: Dec. 11, 2009

Related U.S. Application Data
(60) Provisional application No. 60/934,287, filed on Jun. 11, 2007.

(51) Int. Cl. C12Q 1/68

(52) U.S. Cl. 435/6

(57) ABSTRACT

This document features methods and materials involved in predicting responsiveness of a mammal (e.g., human) having cancer (e.g., glioblastoma multiforme) to treatment with chemotherapy (e.g., temozolomide). For example, methods and materials for using the methylation status at one or more CpG methylation sites (e.g., CpG 89) in O6-methylguanine methyl-transferase nucleic acid to determine whether or not a mammal having cancer is responsive to treatment with chemotherapy (e.g., temozolomide) are provided.

### CROSS-REFERENCE TO RELATED APPLICATIONS

This application claims the benefit of U.S. Provisional Application Ser. No. 60/934,287, filed Jun. 11, 2007. The disclosure of the prior application is incorporated by reference in its entirety.

### STATEMENT AS TO FEDERALLY SPONSORED RESEARCH

This invention was made with government support under grant CA108961 awarded by the National Cancer Institute. The government has certain rights in the invention.

### BACKGROUND

1. Technical Field

This document relates to methods and materials involved in predicting responsiveness of a mammal (e.g., human) having cancer (e.g., glioblastoma multiforme) to treatment with chemotherapy (e.g., temozolomide). For example, this document relates to methods and materials for using the methylation status at one or more CpG methylation sites in O6-methylguanine methyl-transferase nucleic acid to determine whether or not a mammal having cancer is responsive to treatment with chemotherapy (e.g., temozolomide).

2. Background Information

Temozolomide (TMZ) methylates nucleotide bases in genomic DNA, and unrepaired methylation damage of genomic DNA can induce cell death. Common methylation products, such as N7-guanine or N3-adenine can be efficiently repaired through a series of reactions in the base-excision repair (BER) pathway, while O6-methylguanine (O6MG) can be repaired in a one-step process by the O6-methylguanine methyl-transferase (MGMT) DNA repair polypeptide. In an irreversible stoichiometric reaction, the methyl group from O6MG can be covalently transferred to Cys-145 within the active site of MGMT to restore guanine to its native state. Both BER and MGMT activities can be critical for cell survival following methylation damage, and pharmacologic inhibitors of either BER or MGMT repair activities can significantly enhance the cytotoxicity of TMZ by over 50-fold (Trivedi et al., Cancer Res, 65:6394-6400 (2005); Delaney et al., Clinical Cancer Research, 6:2860-2867 (2000); Leuraud et al., Cancer Res, 64:4648-4653 (2004); Barvaux et al., Mol Cancer Ther, 3:1215-1220 (2004); Wedge and Newlands, British Journal of Cancer, 73:1049-1052 (1996); Hirose et al., Journal of Neurosurgery, 98:591-598 (2003); Jean-Claude et al., Journal of Pharmacology & Experimental Therapeutics, 288:484-489 (1999); Hermisson et al., Journal of Neurochemistry, 96:766-776 (2006); Liu and Gerson, Current Opinion in Investigational Drugs, 5:623-627 (2004)).

Unrepaired O6MG can be well tolerated until DNA replication, when O6MG is mispaired with thymine by DNA polymerase. On subsequent rounds of replication, the O6MG:T mispairing can be recognized and bound by the hMsh2 and hMsh6 mismatch repair dimer. Following recruitment of the Mlh1:Pms2 dimer, the repair complex can target the nascent daughter strand, with the mispaired T, for resection and re-synthesis. Since the methylguanine is not removed, thymine is again mispaired with O6MG, and the failure of MMR to resolve the O6MG:T mispair can result in futile cycles of MMR and can ultimately lead to double strand breaks (Karran, Carcinogenesis, 22:1931-1937 (2001); Drablos et al., DNA Repair, 3:1389-1407 (2004)).

### SUMMARY

This document provides methods and materials related to determining the responsiveness of mammals (e.g., humans) having cancer (e.g., glioblastoma multiforme) to treatment with chemotherapy (e.g., temozolomide). For example, this document provides methods and materials for using the methylation status at one or more CpG methylation sites (e.g., CpG 89) in nucleic acid encoding O6-methylguanine methyl-transferase polypeptide to determine whether or not a mammal having cancer is responsive to treatment with chemotherapy (e.g., temozolomide).

This document is based in part on the discovery of CpG methylation sites in nucleic acid encoding an O6-methylguanine methyl-transferase (MGMT) DNA repair polypeptide that are associated with TMZ resistance or sensitivity and survival in mammals having cancer (e.g., GBM). As described herein, the methylation status of one or more CpG methylation sites in MGMT nucleic acid can be assessed in a mammal having cancer, and the methylation status of the one or more CpG methylation sites can be used to determine whether or not the mammal is responsive to chemotherapy (e.g., TMZ) and whether or not the mammal is likely to experience longer survival with chemotherapy treatment than a corresponding mammal that is not responsive to treatment with chemotherapy. For example, the methylation status at CpG 89 can be assessed (e.g., using methylation sequencing) in a tumor (e.g., a tumor biopsy specimen) of a mammal, and methylation at CpG 89 can indicate that the mammal is responsive to treatment with chemotherapy (e.g., TMZ), while lack of methylation at CpG 89 can indicate that the mammal is not responsive to treatment with chemotherapy (e.g., TMZ). In some cases, methylation can be assessed at CpG 8, CpG 58, CpG 66, and CpG 89 in a tumor of a mammal, and methylation at CpG 8, CpG 58, CpG 66, and CpG 89 can indicate that the mammal is responsive to treatment with chemotherapy (e.g., TMZ), while lack of methylation at CpG 8, CpG 58, CpG 66, and CpG 89 can indicate that the mammal is not responsive to treatment with chemotherapy (e.g., TMZ). In some cases, methylation can be assessed at CpG 1, CpG 9, CpG 10, CpG 12, CpG 13, CpG 15, CpG 20, CpG 36, CpG 38, CpG 39, CpG 40, CpG 49, CpG 50, CpG 51, CpG 52, CpG 62, CpG 63, CpG 75, CpG 76, CpG 77, CpG 83, CpG 85, CpG 86, CpG 89, CpG 91, and CpG 93 in a tumor of a mammal, and methylation at CpG 1, CpG 9, CpG 10, CpG 12, CpG 13, CpG 15, CpG 20, CpG 36, CpG 38, CpG 39, CpG 40, CpG 49, CpG 50, CpG 51, CpG 52, CpG 62, CpG 63, CpG 75, CpG 76, CpG 77, CpG 83, CpG 85, CpG 86, CpG 89, CpG 91, and CpG 93 can indicate that the mammal is responsive to treatment with chemotherapy (e.g., TMZ), and lack of methylation at CpG 1, CpG 9, CpG 10, CpG 12, CpG 13, CpG 15, CpG 20, CpG 36, CpG 38, CpG 39, CpG 40, CpG 49, CpG 50, CpG 51, CpG 52, CpG 62, CpG 63, CpG 75, CpG 76, CpG 77, CpG 83, CpG 85, CpG 86, CpG 89, CpG 91, and CpG 93 can indicate that the mammal is not responsive to treatment with chemotherapy (e.g., TMZ).

This document also is based in part on the discovery that the expression level of an MGMT polypeptide in a tumor of a mammal having cancer is inversely proportional to the survival benefit obtained with chemotherapy (e.g., TMZ) treatment. As described herein, the level of MGMT polypeptide expression can be determined in a tumor of a mammal, and the level can be compared to a reference level. A level of MGMT polypeptide expression that is below a reference level can indicate that the mammal is likely to survive longer with chemotherapy (e.g., TMZ) treatment than a mammal (e.g., a mammal of the same species) having a corresponding tumor with an expression level of MGMT polypeptide that is above the reference level.

In general, this document features a method for determining whether or not a mammal having cancer is responsive to treatment with chemotherapy. The method comprises, or consists essentially of, determining the methylation status of CpG 89 in an MGMT nucleic acid in a tumor of the mammal, where methylation at CpG 89 indicates that the mammal is responsive to treatment with chemotherapy, and lack of methylation at CpG 89 indicates that the mammal is not responsive to treatment with chemotherapy. The mammal can be human. The cancer can be brain cancer. The chemotherapy can be temozolomide. The brain cancer can be glioblastoma multiforme. The methylation status of CpG 89 is determined using methylation sequencing. The mammal that is responsive to treatment with chemotherapy and is treated with chemotherapy can be likely to survive longer than a mammal that is not responsive to treatment with chemotherapy and is treated with chemotherapy.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1, panel A contains images of agarose gels analyzing PCR products from MS-PCR analysis of MGMT promoter methylation status in flank tumor specimens used to establish the orthotopic tumors in TMZ therapy evaluations. The lanes corresponding to PCR reactions specific for unmethylated (u) and methylated (m) templates are labeled. For each line, the capitalized letter denotes our interpretation of the methylation status. FIG. 1, panel B contains a Western blot analyzing MGMT protein expression in flank tumor specimens used to establish the orthotopic tumors in TMZ therapy evaluations. The membrane was stripped and re-probed for β-actin.

FIG. 2 is a graph plotting relative survival versus MGMT methylation status as assessed using MS-PCR.

FIG. 3 contains results of MS-sequencing of the MGMT promoter in 12 GBM xenograft lines. Each row represents the results from a single line, and rows are ordered from most TMZ resistant to most TMZ sensitive. Methylation status at each CpG site is shown as methylated (dark gray) or unmethylated (white). MS-PCR results are shown on the far right.

FIG. 4 contains results of Classification and Regression Tree (CART) analysis for prediction of TMZ response based on methylation at specific CpGs.

FIG. 5 lists the methylation status of nine CpG sites as determined by methylation-specific sequencing and MS-PCR for each xenograft line. PC=positive control; NC=negative control; M=methylated; U=unmethylated; H=hemimethylated; 78-82 and 86-89 indicate the nine CpG sites.

FIG. 6 contains images of agarose gels analyzing PCR products from MGMT MS-PCR analysis of multiple xenograft generations for GBM14 and GBM43 as indicated.

FIG. 7, panel A contains a graph plotting normalized luminescence versus days after establishing intracranial tumors with GBM14 cells transduced with a luciferase-expressing lentiviral construct. TMZ treatment was administered on the indicated days. FIG. 7, panel B contains representative bioluminescence imaging results obtained at the indicated time points. FIG. 7, panel C is a graph plotting percent survival versus days after establishing intracranial tumors with GBM14 cells transduced with a luciferase-expressing lentiviral construct. Either a single dose of TMZ was administered, or two doses of TMZ were administered, with the repeat dose being administered at the time of recurrence. TMZ treatment is indicated by arrows.

FIG. 8, panel A contains images of agarose gels analyzing PCR products from MS-PCR analysis of GBM12 parental and derivative resistant tumor lines for methylation. FIG. 8, panel B contains results from MS-sequencing analysis of GBM12 parental and derivative resistant tumor lines for methylation. Hatch marks represent methylated or unmethylated CpG sites, with FIG. 8, panel B of U.S. Provisional Patent Application No. 60/934,287, filed Jun. 11, 2007, having red hatch marks for methylated CpG sites and blue hatch marks for unmethylated sites. There is a region of CpG demethylation shown as outlined. The region interrogated by MS-PCR is also shown.

FIG. 9 contains survival curves for tumors that were the least and most responsive to TMZ. Mice with established orthotopic xenografts were randomized and then treated with placebo or 66 mg/kg TMZ orally for 5 days. TMZ survival was evaluated relative to MGMT methylation status.

FIG. 10, panel A contains a graph plotting relative prolongation in median survival for each tumor line versus MS-PCR methylation status. M—methylated, open circles; U=unmethylated, closed triangles. FIG. 10, panel B contains a graph plotting relative prolongation in median survival for each tumor line versus MGMT polypeptide levels for both methylated (open circles) and non-methylated (closed triangles) tumors. FIG. 10, panel C contains a graph plotting the relative prolongation in median survival for each tumor line versus the methylation status of CpG89. M=methylated, open circles; U=unmethylated, closed triangles.

FIG. 11 contains survival curves for the indicated xenograft lines. Mice with established orthotopic xenografts were randomized and then treated with placebo or 66 mg/kg TMZ orally for 5 days. TMZ survival evaluations were performed in the xenograft panel.

FIG. 12 contains methylation specific DNA sequence data (SEQ ID NOs:19-33, in the order presented). The raw DNA sequence reads for each tumor line are shown. Unmethylated cytosines are converted to uracil and read as thymine (T) during sequencing, while methylated cytosines remain unchanged. The specific CpG sites are numbered on each scan. The wild-type human sequence is as follows: CTTTGCGTCCC-GACGCCCGCAGGTCCTCGCGGTGCGCACCGTTTGCGACTTGGTGAGTGTCTG GGTCGCCTCGCTCCCGGAAGAGTGCGGAG (SEQ ID NO:18).

### DETAILED DESCRIPTION

This document provides methods and materials related to determining the responsiveness of mammals (e.g., humans) having cancer (e.g., glioblastoma multiforme) to treatment with chemotherapy (e.g., temozolomide). For example, this document provides methods and materials for using the methylation status at one or more CpG methylation sites (e.g., CpG 89) in O6-methylguanine methyl-transferase nucleic acid to determine whether or not a mammal having cancer is responsive to treatment with chemotherapy (e.g., temozolomide).

The mammal can be any type of mammal including, without limitation, a mouse, rat, dog, cat, horse, sheep, goat, cow, pig, monkey, or human. The term “MGMT nucleic acid” as used herein refers to a nucleic acid that extends from 5 kb upstream of the transcription start site of MGMT mRNA to 5 kb downstream of the transcription termination site of MGMT mRNA. In some cases, an MGMT nucleic acid can be (1) any nucleic acid that encodes an MGMT polypeptide, (2) any fragment of a nucleic acid that encodes an MGMT polypeptide, (3) any intronic sequences located between exon sequences that encode a portion of an MGMT polypeptide, (4) any 5′ flanking sequence within 5 kb of the transcription start site of MGMT mRNA, (5) any 3′ flanking sequence within 5 kb of the transcription termination site of MGMT mRNA, (6) any sequence located between the transcription start site of MGMT mRNA and the first exon that encodes a portion of an MGMT polypeptide, (7) any sequence located between the last exon that encodes a portion of an MGMT polypeptide and the transcription termination site of MGMT mRNA, or (8) any cis-acting regulatory sequence of MGMT mRNA. An MGMT nucleic acid that encodes an MGMT polypeptide can be, for example, an MGMT mRNA or an MGMT cDNA. In some cases, an MGMT nucleic acid can include an MGMT exon, an MGMT intron, an MGMT 5′ UTR, an MGMT 3′ UTR, and an MGMT promoter sequence as well as sequences encompassing 4 kb, 3 kb, 2 kb, 1 kb, 0.8 kb, 0.5 kb, 0.3 kb, or 0.1 kb upstream of the transcription start site for MGMT mRNA expression and 4 kb, 3 kb, 2 kb, 1 kb, 0.8 kb, 0.5 kb, 0.3 kb, or 0.1 kb downstream of the transcription termination site for MGMT mRNA expression. Examples of MGMT nucleic acid include, without limitation, the nucleic acid sequence set forth in SEQ ID NO:1, the nucleic acid sequence set forth in SEQ ID NO:18, and the nucleic acid sequence set forth in GenBank® under accession number NT_{—}008818.15 (GI:37551413). CpG 1 corresponds to nucleotides 2498808 and 2498809. CpGs are then numbered sequentially as they appear in the sequence through CpG 98, which corresponds with nucleotides 2499623 and 2499624. Using this numbering system, CpG 89 corresponds with nucleotides 2499527 and 2499528, CpG 58 with nucleotides 2499301 and 2499302, CpG 66 with nucleotides 2499366 and 2499367, and CpG 8 with nucleotides 2498907 and 2498908. The nucleic acid set forth in SEQ ID NO:1 corresponds to the sequence from 2498808 to 2499624 and is as follows: CGGCCCCCTGACAGGGTCTCTGCT-GGTCTGGGGGTCCCTGACTAGGGGAGCGGCACCAGGAGGGGAGAGACTCGC GCTCCGGGCTCAGCGTAGCCGCCCCGAGCAGGACCGGGATTCTCACTAAGCG GGCGCCGTCCTACGACCCCCGCGCGCTTTCAGGACCACTCGGGCACGTGGCA GGTCGCTTGCACGCCCGCGGACTATCCCTGTGACAGGAAAAGGTACGGGCCA TTTGGCAAACTAAGGCACAGAGCCTCAGGCGGAAGCTGGGAAGGCGCCGCC CGGCTTGTACCGGCCGAAGGGCCATCCGGGTCAGGCGCACAGGGCAGCGGC GCTGCCGGAGGACCAGGGCCGGCGTGCCGGCGTCCAGCGAGGATGCGCAGA CTGCCTCAGGCCCGGCGCCGCCGCACAGGGCATGCGCCGACCCGGTCGGGCG GGAACACCCCGCCCCTCCCGGGCTCCGCCCCAGCTCCGCCCCCGCGCGCCCC GGCCCCGCCCCCGCGCGCTCTCTTGCTTTTCTCAGGTCCTCGGCTCCGCCCCG CTCTAGACCCCGCCCCACGCCGCCATCCCCGTGCCCCTCGGCCCCGCCCCCGC GCCCCGGATATGCTGGGACAGCCCGCGCCCCTAGAACGCTTTGCGTCCCGAC GCCCGCAGGTCCTCGCGGTGCGCACCGTTTGCGACTTGGTGAGTGTCTGGGTC GCCTCGCTCCCGGAAGAGTGCGGAGCTCTCCCTCGGGACGGTGGCAGCCTCG AGTGGTCCTGCAGGCGCCCTCACTTCGCCGTCGGGTGTGGGGCCGCCCTGAC CCCCACCCATCCCG.

Any appropriate method can be used to assess the methylation status at one or more CpG sites in an MGMT nucleic acid. For example, a method described herein, such as methylation sequencing or deep-amplicon sequencing can be used. In addition, any appropriate method, such as a method described herein, can be used to determine the level of MGMT polypeptide expression in a tumor of a mammal. Methylation status and MGMT polypeptide expression level in a tumor of a mammal can be assessed by analyzing any appropriate sample of a tumor from the mammal. For example, a tumor biopsy specimen can be analyzed to determine the methylation status at one or more CpG sites in an MGMT nucleic acid or to determine an MGMT polypeptide expression level.

The term “reference level” as used herein with respect to an MGMT polypeptide is the level of the MGMT polypeptide typically expressed by mammals free of cancer. For example, a reference level of an MGMT polypeptide can be the median level of the MGMT polypeptide that is present in samples obtained from a random sampling of humans that are free of cancer. Control samples used to determine a reference level can be obtained from any appropriate number of mammals (e.g., 10, 20, 30, 40, 50, 75, 100, 125, 150, 175, 200, 250, 300, 400, 500, 600, 700, 800, 900, 1000 or more mammals) from the same species as the mammal being evaluated. In some cases, control samples can be obtained from humans of the same race, age group, and/or geographic location as the mammal being evaluated.

This document also provides methods and materials related to detecting clones that are resistant to chemotherapy (e.g., TMZ) in a tumor (e.g., glioblastoma multiforme) from a mammal. For example, the methylation status at one or more CpG methylation sites in O6-methylguanine methyl-transferase nucleic acid can be used to identify clones that are resistant to chemotherapy (e.g., TMZ) in a tumor from a mammal. In some cases, the presence of one or more mutations or polymorphisms in a mismatch repair nucleic acid can be used to identify clones that are resistant to chemotherapy (e.g., TMZ) in a tumor from a mammal (e.g., human). Mutations or polymorphisms can be detected using any appropriate method, such as deep-amplicon sequencing. The invention will be further described in the following examples, which do not limit the scope of the invention described in the claims.

### EXAMPLES

### Example 1

### Using a Xenograft Model to Evaluate TMZ Responsiveness

The Neuro-Oncology program at Mayo Clinic has developed and maintained a panel of human GBM xenografts through direct implantation and propagation of patient tumor samples in nude mice. The xenograft lines have been used to establish orthotopic tumors for evaluation of TMZ responsiveness. A subset of the xenografts were derived from patients who were treated with TMZ during their clinical care. There was a good correlation between the observed clinical and xenograft responses to TMZ treatment in the lines. Similar to clinical observations, TMZ sensitivity in all xenograft lines tested roughly correlated with MGMT promoter methylation status, although the predictive accuracy of the methylation-specific PCR (MS-PCR) assay was marginal. Through a comprehensive evaluation of CpG methylation in the xenografts, a more robust model emerged for predicting TMZ response based on the methylation status of four distinct CpG methylation sites across the MGMT promoter. A similar analysis is performed in clinical samples to develop a robust model for predicting TMZ sensitivity.

The xenograft model also provides opportunities to study how changes in MGMT promoter methylation affect TMZ sensitivity. With prolonged serial tumor passage in mice, corresponding changes in TMZ sensitivity and MGMT polypeptide expression were observed. This effect may be due to a progressive change in MGMT promoter methylation patterns. Using the xenograft model in conjunction with an MGMT promoter-luciferase reporter assay, key CpG methylation sites that are important for regulating MGMT expression and TMZ sensitivity are independently confirmed.

The ability to access tumor following treatment provides a powerful tool for studying the evolution of drug resistance during TMZ therapy. Rapid development of TMZ resistance during therapy was observed in some of the xenograft lines. Resistance may arise from the selection of pre-existing tumor clones harboring MGMT promoter methylation patterns conferring TMZ resistance. Advanced pyrosequencing techniques are used to test whether the pre-existing clones can be detected in previously untreated tumors. The xenograft studies compliment clinical studies to examine the relationship between MGMT promoter methylation, TMZ responsiveness, and the development of resistance.

Experiments are performed to identify MGMT promoter methylation patterns associated with improved survival in newly diagnosed GBM patients treated with a TMZ-based therapy. CpG methylation patterns are evaluated by bisulfite sequencing and are compared to MGMT MS-PCR results in patient tumor samples collected in two prospective clinical trials. The methylation pattern model subsequently is validated in a set of patient samples from a third independent clinical trial. Changes in TMZ sensitivity and MGMT methylation patterns are correlated in early passage versus late passage xenografts. From an archive of tumor tissue for each xenograft line, changes in MGMT methylation patterns are correlated with MGMT expression levels, and cryopreserved tumor lines are restored to allow evaluation of TMZ responsiveness at multiple generations. Mechanisms of acquired resistance during cyclical TMZ therapy are evaluated. The mechanism(s) of TMZ resistance development is studied in several xenograft lines by analyzing recurrent orthotopic tumors from mice treated with placebo or one or more cycles of TMZ therapy.

Serially-transplanted xenograft model for therapy evaluation: Pre-clinical therapeutic testing traditionally has been performed using tumor cell lines that have been selected for and maintained in cell culture for many years. As a consequence, the genetic and morphologic characteristics of these tumors may not accurately reflect those typically found in primary human tumors. In GBM, an example of this is the lack of tumorigenic cell lines that harbor amplified EGFR despite this genetic lesion being found in approximately 40% of primary GBM tumor specimens (Smith et al., J Natl Cancer Inst, 93:1246-1256 (2001); Frederick et al., Cancer Res, 60:1383-1387 (2000)). Prolonged cell culture can lead to progressive hypermethylation of the MGMT promoter (Pegg, Cancer Research, 50:6119-6129 (1990); Yamada et al., Journal of Biological Chemistry, 276:19052-19058 (2001); Danam et al., International Journal of Oncology, 18:1187-1193 (2001)), and about 80% of glioma cell lines are MGMT hypermethylated, as compared to a 45% incidence of promoter hypermethylation in clinical samples. A panel of 17 GBM xenografts was developed. The xenografts were established by implanting patient tumor specimens directly into the flank of nude mice. Maintenance of these xenograft lines by serial transplantation in mice can preserve genetic features of the corresponding patient tumor, including EGFR amplification (Pandita et al., Genes Chromosomes & Cancer, 39:29-36 (2003)). When transplanted into the brain, these xenograft lines can form tumors with histopathologic features commonly seen in primary GBM tumors (Giannini et al., Neuro-Oncology, 7:164-176 (2005)). Using this panel of xenografts and an intracranial tumor therapy model, it was evaluated whether EGFR amplification is associated with intrinsic radiation resistance. Consistent with a recent analysis of patients enrolled on RTOG studies, it was demonstrated that EGFR amplification had no influence on intrinsic radiation response: 12 Gy significantly enhanced the survival of four of seven xenografts with EGFR amplification and two of six without EGFR amplification. In a similar analysis for sensitivity to EGFR inhibitors, survival of two of 11 xenograft lines was significantly prolonged following chronic therapy with erlotinib. Consistent with the results of clinical studies (Mellinghoff et al., N Engl J Med, 353:2012-2024 (2005); Haas-Kogan et al., J Natl Cancer Inst, 97:880-887 (2005)), both xenograft lines identified as being sensitive to erlotinib harbor amplifications of either wild-type EGFR (GBM12) or the vIII mutant EGFR (GBM39), and both lines have wild-type PTEN with low phospho-Akt activity. The efficacy of TMZ was evaluated in multiple xenograft lines, and in comparison to novel therapeutic agents such as erlotinib or RAD001, TMZ had profound effects on survival in specific xenograft lines that exceeded those seen with other therapies except radiation (Table 1). It is demonstrated herein that the xenograft panel is a highly relevant model system, specifically for evaluating mechanisms of sensitivity and resistance to TMZ-based therapies.

TABLE 1
Results of survival analyses with TMZ treatment in 17 GBM
xenograft lines in an orthotopic treatment model

| | [symbol] pla- | | | | | | |
|---|---|---|---|---|---|---|---|
| | cebo | median | TMZ | median | Survival | log-rank | MS- |
| GBM | n = | survival | n = | survival | benefit | p = | PCR |
| 6 | 8 | 41 | 8 | 56 | 37% | 0.019 | U |
| 8 | 8 | 59 | 8 | 124 | 110% | <0.001 | M |
| 10 | 7 | 41 | 9 | 55 | 34% | 0.362 | U |
| 12 | 10 | 15 | 10 | 53 | 253% | <0.001 | M |
| 14 | 10 | 33 | 10 | 154 | 558% | <0.001 | U |
| 16 | 8 | 47 | 8 | 300 | 579% | 0.021 | M |
| 22 | 8 | 43 | 8 | 124 | 260% | <0.001 | M |
| 26 | 8 | 83 | 8 | 100 | 21% | 0.003 | U |
| 28 | 8 | 33 | 8 | 56 | 70% | 0.013 | U |
| 34 | 8 | 89 | 9 | 351 | 294% | <0.001 | U |
| 36 | 8 | 126 | 8 | 214 | 82% | 0.142 | M |
| 38 | 9 | 55 | 10 | 80 | 45% | <0.001 | U |
| 39 | 10 | 31 | 10 | >104 | >235% | NA | M |
| 43 | 7 | 23 | 8 | 43 | 87% | <0.001 | U |
| 44 | 9 | 35 | 10 | 56 | 60% | 0.336 | U |
| 46 | 9 | 50 | 9 | 50 | 0% | 0.332 | U |
| 59 | 9 | 46 | 9 | 90 | 96% | <0.001 | U |

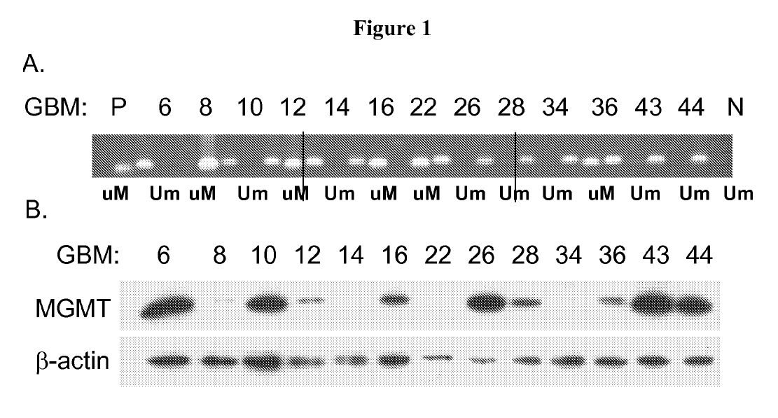

MGMT methylation and expression status: MGMT promoter methylation and MGMT polypeptide expression status have been implicated in TMZ sensitivity. To validate these measures as potential predictors of TMZ sensitivity, MGMT promoter methylation was evaluated by bisulfite methylation-specific PCR and MGMT polypeptide expression was evaluated by Western blotting in flank tumor specimens from the xenograft panel. In an initial analyses in 13 xenograft lines, five specimens (38%) tested positive for MGMT promoter methylation (FIG. 1), which is a frequency similar to that seen in clinical GBM specimens (Hegi et al., N Engl J Med, 352:997-1003 (2005); Esteller et al., N Engl J Med, 343:1350-1354 (2000); Hegi et al., Clin Cancer Res, 10:1871-1874 (2004); Esteller et al., Cancer Res, 59:793-797 (1999)). Consistent with observations demonstrating heterogeneous MGMT promoter methylation, PCR amplification yielded product in reactions specific for methylated and unmethylated templates in four xenograft lines (GBM8, 12, 16 and 36) and for methylated templates alone in one xenograft line (GBM22). With respect to the effects of promoter methylation on polypeptide expression, tumor samples with evidence of MGMT promoter methylation uniformly had low levels of MGMT polypeptide. In contrast, a significant degree of variation in MGMT expression was evident in non-methylated tumors, which ranged from robust expression (GBM10 and 43) to undetectable (GBM14 and 34).

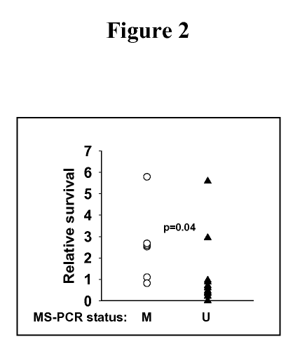

In vivo sensitivity of GBM lines to TMZ: To address whether MGMT methylation or polypeptide expression status are predictive of TMZ responsiveness, the effects of TMZ therapy on survival were evaluated in the panel of xenografts using an orthotopic therapy model. Mice with established intracranial xenografts were treated with 66 mg/kg TMZ or placebo daily for 5 days by oral gavage. This TMZ dosing regimen provides the equivalent dose-exposure to TMZ as the standard adjuvant TMZ dosing regimen for GBM patients of 200 mg/m²×5 days. All mice were followed until reaching a moribund state, and the relative prolongation of survival following TMZ treatment was calculated for each line relative to placebo treatment (Table 1). For the MGMT methylated tumors, TMZ therapy extended the median survival of mice by 110% (GBM8) to as much as 579% (GBM16). In general, TMZ was less effective in the non-methylated MGMT tumors, although the range of survival prolongation was highly variable between non-methylated cases, with the lowest and highest survival benefit associated with GBM26 (21%) and GBM14 (558%), respectively. Further analysis of the relationship between TMZ responsiveness and MGMT methylation status in 17 xenograft lines was conducted by comparing the distribution of median survival prolongation results for methylation-positive and methylation-negative groups of xenografts. From this analysis, MGMT methylation was modestly associated with increased TMZ sensitivity (FIG. 2, p=0.039, two-sample rank sum test). In a similar analysis of MGMT polypeptide expression levels, high levels of MGMT polypeptide expression were invariably associated with relative TMZ resistance, and the absence of MGMT polypeptide expression was associated with significant TMZ sensitivity (r=−0.73, p=0.005). For tumors with low but detectable levels of MGMT polypeptide expression (GBM12, 16, 28, and 36), there was considerable variability in the survival benefit ranging from 70% to 579% prolongation in median survival. While p53, PTEN and EGFR function have been linked to TMZ sensitivity in some models, no correlation between genetic alterations of these genes and TMZ responsiveness was observed. Collectively, these data demonstrate that MGMT promoter methylation or low levels of MGMT polypeptide expression are associated with higher efficacy of TMZ therapy, but that similar to clinical experience, there is a relatively wide-range in sensitivity associated with either marker of TMZ sensitivity.

Association between xenograft and patient response to TMZ: Five of the patients from whom xenograft lines were established were treated with TMZ at some point during the course of their disease, which allows for a direct comparison between xenograft and clinical responsiveness to TMZ. Four of the xenografts were established from resection material obtained at the time of initial diagnosis (GBM26, 34, 38, and 39) and one xenograft line was established from tumor obtained following recurrence (GBM46). All patients were treated with radiation monotherapy and received TMZ at the time of recurrence. Consistent with the relative resistance to TMZ observed in xenografts lines GBM26 (21% prolongation in survival) and GBM38 (45%), both patients from whom these tumors were derived rapidly progressed on their second cycle of TMZ therapy. TMZ treatment of mice with GBM34 xenografts resulted in moderate improvement in survival (294%). The corresponding patient failed an initial cycle with BCNU but then had stable disease for three cycles of TMZ. Similarly, the GBM39 xenograft was responsive to TMZ (median survival in TMZ-treated mice not yet reached, survival prolongation at least 235%), and the corresponding patient had an initial partial response to TMZ with subsequent progression after seven cycles of drug therapy. GBM46 (0% prolongation of survival) was derived from a tumor that was resected following progression on TMZ therapy. This patient was initially treated with radiation and erlotinib. At the time of initial progression, the patient received three cycles of BCNU with initially stable disease. With progression, the patient was switched to TMZ 150 mg/m²×5 every 28 days, but rapidly progressed during the first cycle and was taken for re-resection, at which time the tumor sample used to derive GBM46 was obtained. These results indicate that TMZ responsiveness of individual tumor lines in the therapy evaluation model correlate closely with the clinical assessment of TMZ responsiveness in the corresponding patients.

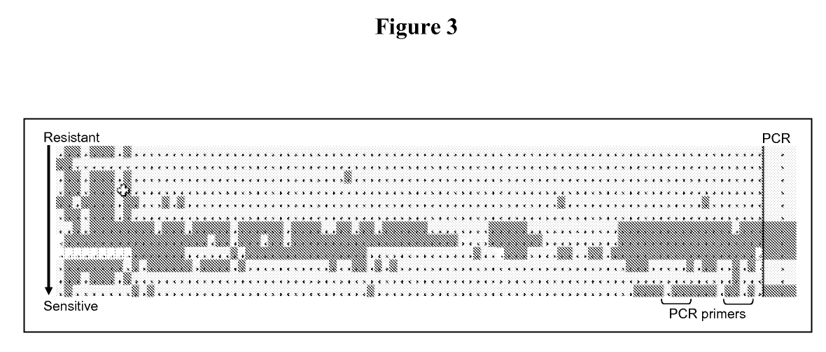

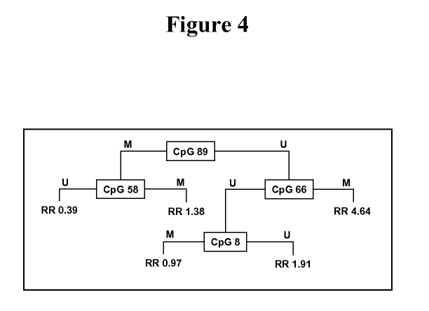

MGMT CpG methylation sequencing: The MGMT CpG island contains 98 CpG sites, and a comprehensive evaluation of CpG methylation patterns within this region may be more predictive of TMZ sensitivity than MS-PCR. Numbering the CpG sites from 5′ to 3′, the primers for the widely used MS-PCR analysis of MGMT methylation anneal across CpG sites 78-82 and 86-89. The methylation status of each CpG site within the entire MGMT CpG island was determined by DNA sequencing following bisulfite modification in 12 of the xenograft lines (FIG. 3), and these data were correlated with TMZ responses measured in the xenograft model using a CART analysis. In this analysis, a predictive model for TMZ sensitivity based on methylation at four CpG sites was identified (FIG. 4). In a proportional hazards regression model, the hazard ratios associated with methylation at the four sites were: CpG 89—HR 0.21 (p<0.001), CpG 58—HR 3.26 (p=0.05), CpG 66—HR 11.55 (p<0.001), CpG 8—HR 0.32 (p−0.027). In comparison, the hazard ratio for the MS-PCR assay was 0.77 (p=0.241), which suggests that the four site methylation model provides a more robust model of TMZ sensitivity as compared to MS-PCR. These results suggest that methylation at individual CpG sites distinct from those queried by the MS-PCR assay are important predictors of TMZ responsiveness.

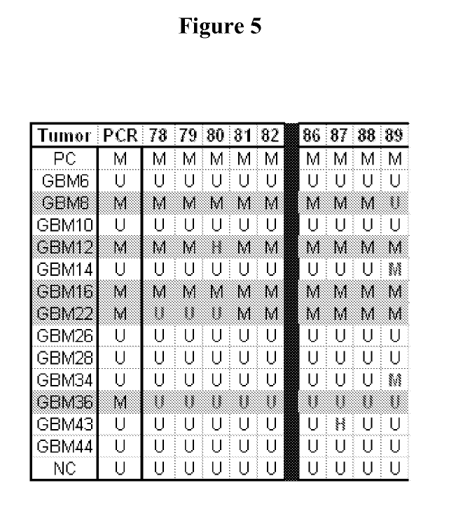

The MS-PCR assay specifically evaluates the methylation status of CpGs 78-82 and 86-89. The MS-PCR results and the sequencing results for these specific CpGs were compared. In GBM36, which is methylated by MS-PCR, there was discordance between the two methods, with all sites found to be unmethylated by MS-sequencing. For the other lines, there was concordance between MS-PCR and MS-sequencing results for CpG 81, 82, 86, and 88. For the other five sites, methylation status defined by MS-PCR and by sequencing was discordant in at least one line (FIG. 5). CpG 89 was the most divergent between the MS-PCR and sequencing assessments. Interestingly, in the original CART analysis and in a CART analysis limited to the nine CpG sites, the methylation status of CpG 89 was identified as being the most important discriminator between sensitive and resistant tumors (HR 0.16 for a single site model based on CpG 89, p<0.001). In comparing the three models using the Akaike information criterion (AIC) measure for goodness-of-fit in the proportional hazards regression model, the four site model provided the best fit to the data (AIC 596), followed by the single site CpG 89 model (AIC 682), and both of these models were superior to the MS-PCR model (AIC 728). These data highlight the potential utility of analyzing the methylation status of individual CpG sites within the MGMT promoter and suggest that such an approach will provide a more robust predictor of TMZ responsiveness when applied to clinical specimens.

In addition to the CART analysis, the correlation between methylation at any given CpG site and TMZ responsiveness was evaluated. The following analyses pool GBM tumor lines 6, 8, 10, 12, 14, 16, 22, 26, 28, 34, 36, 43, and 44 for TMZ treated mice only to identify any potential univariate association of CpG site with survival. This analysis was completed using proportional hazards regression. Hazard ratios<1 indicate better survival. All results where p-values were <0.05 are included.

CpG 1: HR=6.21; 95% CI: 3.24-11.92; p<0.001;
CpG 9: HR=3.59; 95% CI: 1.94-6.66; p<0.001;
CpG 10: HR=0.51; 95% CI: 0.33-0.80; p=0.003;
CpG 12: HR=0.35; 95% CI: 0.22-0.56; p<0.001;
CpG 13: HR=0.61; 95% CI: 0.38-0.99; p=0.045;
CpG 15: HR=0.64; 95% CI: 0.41-0.99; p=0.045;
CpG 20: HR=0.38; 95% CI: 0.21-0.68; p=0.001;
CpG 36: HR=0.61; 95% CI: 0.38-0.99; p=0.045;
CpG 38: HR=1.56; 95% CI: 1.01-2.41; p=0.045;
CpG 39: HR=0.61; 95% CI: 0.38-0.99; p=0.045;
CpG 40: HR=0.64; 95% CI: 0.41-0.99; p=0.045;
CpG 49: HR=3.79; 95% CI: 1.83-7.85; p<0.001;
CpG 50: HR=3.79; 95% CI: 1.83-7.85; p<0.001;
CpG 51: HR=3.79; 95% CI: 1.83-7.85; p<0.001;
CpG 52: HR=3.79; 95% CI: 1.83-7.85; p<0.001;
CpG 62: HR=3.79; 95% CI: 1.83-7.85; p<0.001;
CpG 63: HR=3.79; 95% CI: 1.83-7.85; p<0.001;
CpG 75: HR=0.64; 95% CI: 0.41-0.99; p=0.045;
CpG 76: HR=0.35; 95% CI: 0.22-0.56; p<0.001;
CpG 77: HR=0.35; 95% CI: 0.22-0.56; p<0.001;
CpG 83: HR=0.32; 95% CI: 0.20-0.50; p<0.001;
CpG 85: HR=0.45; 95% CI: 0.29-0.69; p<0.001;
CpG 86: HR=0.32; 95% CI: 0.20-0.50; p<0.001;
CpG 89: HR=0.13; 95% CI: 0.07-0.23; p<0.001;
CpG 91: HR=0.45; 95% CI: 0.29-0.68; p<0.001;
CpG 93: HR=0.48; 95% CI: 0.25-0.92; p=0.027.

Methylation of CpG 58, CpG 66, and CpG 8, which were identified in the CART model, were not significant based on a proportional hazards regression model:

CpG 58: HR=1.57; 95% CI: 0.92-2.68; p=0.102;
CpG 66: HR=1.50; 95% CI: 0.87-2.58; p=0.144;
CpG 8: HR=0.66; 95% CI: 0.40-1.07; p=0.088.

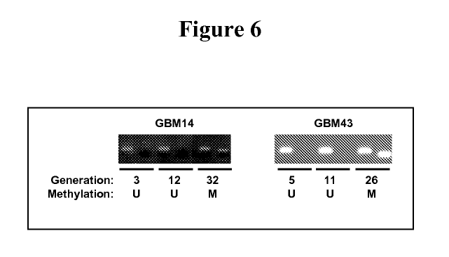

MGMT methylation, MGMT expression, and TMZ sensitivity with extended xenograft passage: Repeat survival experiments with TMZ have been performed in six xenograft lines. In initial studies, a TMZ dosing schedule of 120 mg/kg for 5 days was used. Subsequently, these studies were performed with a dose of 66 mg/kg for 5 days (Table 2). In several tumor lines, the observed survival prolongation for mice with intracranial tumors was greater in the latter experiments in which a lower dose of TMZ was used (GBM10, GBM14 and GBM44). These observed differences in survival benefit were only observed in non-methylated MGMT tumors and only when the tumors used in the survival studies were separated by seven or more generations (passages). To evaluate whether changes in MGMT expression might account for the observed changes in TMZ sensitivity, flank tumor samples from multiple generations of GBM14 and GBM43 were processed for MGMT promoter methylation analysis. This analysis demonstrated clear development of MGMT promoter hypermethylation (by MS-PCR) in late generation GBM14 and 43 tumors (FIG. 6). These data suggest that extended propagation of the GBM xenografts by serial flank transplantation may lead to progressive methylation and silencing of MGMT expression. Studies described elsewhere have suggested that MGMT silencing results in a mutator phenotype, which promotes the accumulation of genomic mutations involved in tumor progression (Allay et al., Oncogene, 18:3783-3787 (1999); Park et al., Cancer, 92:2760-2768 (2001); Dumenco et al., Science, 259:219-222 (1993)). Since any xenograft generation can be restored from cryopreserved material, these observations provide an opportunity to study how progressive epigenetic changes influence TMZ responsiveness and the malignant phenotype within individual xenograft lines.

TABLE 2
Survival benefit with TMZ therapy relative to tumor
generation used to establish the experiment

| | | 120 mg/kg | Survival | 66 mg/kg | Survival | Current |
|---|---|---|---|---|---|---|
| MS-PCR | GBM | Gen. # | benefit | Gen. # | benefit | Gen. # |
| U | 6 | 20 | 67% | 24 | 21% | 38 |
| U | 10 | 8 | 6% | 19 | 34% | 29 |
| M | 12 | 10 | 268% | 30 | 164% | 40 |
| U | 14 | 21 | 148% | 33 | 421% | 44 |
| U | 43 | 6 | 104% | 10 | 83% | 32 |
| U | 44 | 5 | −7% | 12 | 60% | 17 |

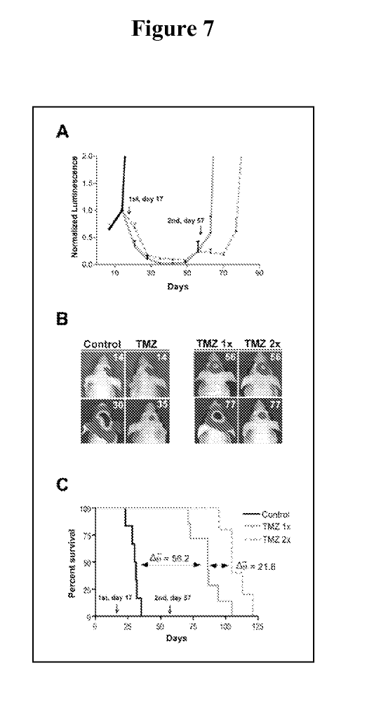

Development of resistance to TMZ during in vivo therapy: The effect of salvage TMZ therapy in recurrent tumors was studied in the GBM14 xenograft line with the assistance of bioluminescence imaging (BLI). In this study, GBM14 cells were transduced with a luciferase-expressing lentiviral construct and then used to establish intracranial tumors. Mice were randomized into three groups: a) vehicle control, b) single dose of 120 mg/kg TMZ, c) initial TMZ dose followed by a second TMZ dose at the time of tumor regrowth, as indicated by BLI (FIGS. 7A and B). Interestingly, even though the second TMZ dose was administered to mice when they had a lower luminescent signal than at initial dosing, the second dose only extended survival by an additional 22 days beyond the 56 day survival prolongation achieved with a single TMZ dose (FIG. 7C). These data suggest that even a single dose of TMZ can lead to selection of TMZ resistant tumor clones which limit the efficacy of subsequent cycles of therapy. Extrapolating to the clinic, the apparent development of TMZ resistance during the course of treatment is reminiscent of patients who experience an initial response to TMZ therapy but then develop progressive disease during latter cycles of TMZ treatment. These results indicate that a xenograft model can be a highly clinically relevant tumor model system for studying the mechanism(s) of TMZ resistance since the model provides ready access to pre- and post-treatment tissue specimens.

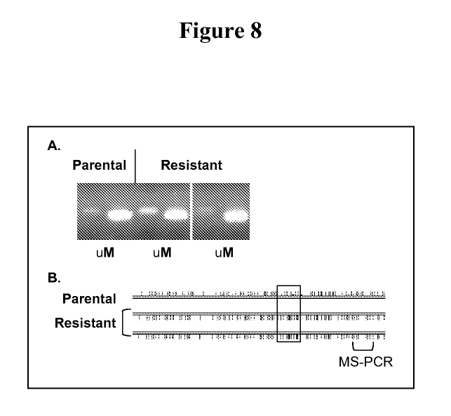

TMZ resistance development associated with changes in MGMT promoter methylation: A TMZ-resistant xenograft line was developed through serial in vivo treatment with TMZ. Preliminary characterization of this tumor line suggests that resistance is associated with alterations of MGMT methylation. To develop the tumor line, a single 1 cm³ GBM12 flank tumor was treated with three doses of 20 mg/kg TMZ over one week. Following an initial complete regression, the recurrent tumor was insensitive to a re-challenge with 66 mg/kg TMZ×3 doses. Samples of this tumor were frozen for analysis, and the tumor was passaged into a second mouse. Once the tumor was approximately 1 cm³, rechallenge with 120 mg/kg TMZ was completely ineffective, and the progressive tumor was frozen for analysis. Although an initial analysis by MS-PCR suggested no difference in methylation status between the parental and derivative resistant lines (FIG. 8A), subsequent CpG methylation sequencing revealed a change in the methylation status of multiple CpG sites outside of the nine CpG sites interrogated by the MS-PCR (FIG. 8B). Interestingly, one of the altered CpG sites (CpG 58) also was identified in the CART analysis of untreated tumors as being important for predicting TMZ responsiveness (FIG. 4). Analysis of the same tumor specimens demonstrated that the altered MGMT methylation patterns were associated with elevated expression of MGMT polypeptide levels. Given the relatively rapid emergence of apparent TMZ resistance in this experiment and the experiment with GBM14 described above, resistance may develop due to selection of pre-existing tumor clones that are inherently resistant to TMZ. Heterogeneous methylation patterns within the MGMT promoter have been reported, and the change in MGMT methylation pattern following TMZ treatment may have arisen due to a selection of tumor cells with a ‘resistant’ MGMT methylation pattern.

These results demonstrate that the relationship between CpG methylation patterns and TMZ sensitivity is complex, and a comprehensive evaluation of MGMT promoter CpG methylation is likely to be more informative than the relatively crude evaluation of methylation by MS-PCR. The methylation sequence data demonstrate significant heterogeneity in CpG methylation patterns across the MGMT promoter in different tumors.

### Example 2

### Identifying CpG Methylation Patterns in the MGMT Promoter that can Predict TMZ Sensitivity

Experiments are performed to determine whether a comprehensive evaluation of CpG methylation patterns within the MGMT promoter in patient tumor samples can provide a more robust predictor of TMZ sensitivity than MS-PCR. Experiments are performed using samples from three studies in which patients have been treated with TMZ and RT.

The GBM xenograft model described herein is a highly relevant model system for understanding the influence of MGMT promoter hypermethylation on TMZ response. Similar to clinical observations, tumors with MGMT promoter hypermethylation (assessed by MS-PCR) were generally more sensitive to TMZ than tumors lacking MGMT methylation, although there was a broad range in sensitivities in both methylated and unmethylated tumors. In a subset of tumors, there was a good correlation between TMZ responsiveness observed in patients and their derivative xenograft lines. Also similar to clinical observations, apparent TMZ resistance developed relatively quickly during TMZ therapy in at least two of the xenograft lines. By analyzing an untreated parental and derivative resistant tumor line, it was observed that the development of TMZ resistance was associated with discrete changes in the MGMT methylation at specific CpG sites. In addition, it was observed that long-term propagation of tumors as flank xenografts in the model can result in increasing TMZ responsiveness that may be due to progressive hypermethylation within the MGMT promoter. The link between changes in methylation at specific CpG sites and changes in TMZ sensitivity is explored in the xenograft model. Changes in MGMT CpG methylation patterns, which occur spontaneously during serial passage, are correlated with changes in TMZ response to understand further how CpG methylation changes influence TMZ response in near-isogenic systems. In addition, experiments are performed to test whether TMZ therapy leads to induction of resistance through selection of tumor clones with MGMT methylation patterns conferring TMZ resistance. Pyrosequencing technology is used to determine whether these selected ‘resistant’ MGMT methylation patterns can be detected in tumor samples prior to therapy.

Identifying MGMT Promoter Methylation Patterns Associated with Improved Survival in Newly Diagnosed GBM Patients Treated with a TMZ-Based Therapy

About 30% of patients with MGMT hypermethylation progress while on TMZ therapy, and about 15% of patients lacking MGMT hypermethylation have prolonged survival with TMZ treatment. The MGMT CpG island spans from −552 to +289 nucleotides (relative to the transcriptional start site) and encompasses 98 CpG sites. The primers for the MGMT MS-PCR assay anneal to two DNA sequences that contain a total of nine CpG sites near the transcriptional start site (Esteller et al., Cancer Res, 59:793-797 (1999)). Relatively subtle changes in CpG methylation at sites distant from this region can have significant effects on MGMT expression (Patel et al., Molecular & Cellular Biology, 17:5813-5822 (1997); Watts et al., Molecular & Cellular Biology, 17:5612-5619 (1997)). Evaluation of MGMT CpG island methylation in the xenograft model suggested that methylation at CpG sites beyond those queried by MS-PCR are important predictors of TMZ responsiveness (FIGS. 3, 4, and 8). Specific patterns of CpG methylation within the MGMT promoter may provide a more robust predictor of TMZ sensitivity than MGMT MS-PCR.

To determine whether specific patterns of CpG methylation within the MGMT promoter can provide a more robust predictor of TMZ sensitivity than MGMT MS-PCR, experiments are performed using tumor specimens obtained from patients in three prospective clinical trials performed by the North Central Cancer Treatment Group (N0177), the University of California, San Francisco (UCSF OSI 2725s), and the Radiation Therapy Oncology Group (RTOG 0525). The analysis of tumor samples from the two smaller NCCTG and UCSF trials is combined to serve as a training set, and tumor samples from the RTOG trial are used as a validation set for the analysis of MGMT methylation patterns. The NCCTG and UCSF trials are Phase 2 studies evaluating the combination of RT/TMZ with erlotinib, an oral EGFR inhibitor. The treatment regimens in these two trials are very similar to the standard regimen of 60 Gy 3D-conformal radiation and continuous TMZ followed by six cycles of adjuvant TMZ given five days out of 28 days, and daily oral erlotinib given continuously during both the RT/TMZ and the adjuvant TMZ. The RTOG 0525 trial is a Phase 3 trial in which patients are treated with the standard regimen of 60 Gy 3D-conformal radiation and continuous TMZ and randomized between standard adjuvant TMZ (as was described for the other two trials) and dose-dense TMZ therapy. Because the relationship between TMZ responsiveness and MGMT methylation patterns may be different for the experimental dose-dense regimen, only those patients enrolled in R0525 who are randomized to standard TMZ treatment are used in the analysis. The TMZ therapy regimens between N0177, UCSF OSI 2725s, and the standard adjuvant TMZ arm of RTOG 0525 are highly similar and form the basis for the analyses.

A combined total of 191 patients have been treated in the NCCTG and UCSF trials, and tumor tissue has been collected for most patients. Combining samples from both studies can provide between 87 and 134 samples with sufficient tumor volume for the molecular analyses, and these samples are used as a training set to evaluate the influence of MGMT CpG methylation on TMZ responsiveness. Studies described elsewhere have suggested that EGFR amplification and wild-type PTEN function are important predictors of erlotinib sensitivity (Mellinghoff et al., N Engl J Med, 353:2012-2024 (2005), Haas-Kogan et al., J Natl Cancer Inst, 97:880-887 (2005)), and an analysis of these molecular factors is performed for the patient specimens collected in N0177, and parallel analyses are performed for the UCSF data set. Molecular analyses of predictors for erlotinib efficacy may be meaningful when performed in the context of an analysis of MGMT. Therefore, MS-sequencing is evaluated as a potential predictor of TMZ sensitivity, and this analysis is combined with data pertaining to EGFR amplification status and PTEN function to provide a comprehensive evaluation of potential predictors of response to erlotinib, radiation, and TMZ.

The results from the NCCTG and UCSF training sets are validated using patient tumor samples obtained in RTOG 0525. Only patients treated using the conventional TMZ adjuvant dosing schedule of RTOG 0525, similar to those used in the other two trials, are analyzed in this study. Over 400 patients are treated in this trial with standard adjuvant TMZ, and tumor samples from the first 200 patients enrolled on this treatment arm are used in the analysis. All patients accrued onto this study are required to have undergone a tumor resection and submission of tumor tissue is required for participation. A goal of these studies is to better understand how MGMT methylation patterns within the promoter region correlate with survival following TMZ-based therapies. When MGMT methylation patterns are observed to provide a superior predictor of TMZ response as compared to conventional MS-PCR, then this information is used to develop an assay that is amenable to high-throughput routine analysis of tissue specimens.

To directly test whether MS-sequencing can provide a more robust predictor of TMZ response than MS-PCR, the following studies are preformed: a) quantitative methylation-specific PCR, b) methylation-specific sequencing of the MGMT CpG island, and c) statistical correlation of patient outcome with methylation status.

Quantitative MGMT MS-PCR: Methylation-specific PCR is a rapid assay for examining the methylation status of selected CpG sites. In this assay, DNA is extracted from paraffin-embedded tissues, denatured with NaOH, and then treated with sodium bisulfite. The chemical reaction modifies non-methylated cytosine to uracil. The resulting methylation-specific DNA sequence alterations can be assessed with two sets of PCR primers specifically designed to anneal to DNA sequences derived from either a methylated or unmethylated template. The technique evaluates the methylation status of those CpG sites contained within the PCR primer sequences. The primers used in the MS-PCR assay for MGMT are specifically designed to encompass a region near the transcription initiation site (Esteller et al., Cancer Res, 59:793-797 (1999)). MS-PCR was used to assess MGMT promoter methylation status in the xenograft lines (FIG. 3). In general, there was a reasonable agreement between methylation status and tumor sensitivity to TMZ treatment (Table 1). As an improvement on this technique, a quantitative MS-PCR assay was developed, and this assay is used to analyze the patient samples. Based on the xenograft data and the EORTC data, the extent of MGMT methylation detected by this technique may correlate with improved survival, but a significant fraction of patients with MGMT promoter methylation may progress while on TMZ therapy and another subset of patients with non-methylated MGMT tumors may experience prolonged survival.

The amount of genomic DNA required for methylation analysis precludes using stereotactic biopsy samples. About 30% of patients enrolled in NCCTG or UCSF trials have had needle biopsies, and not every patient enrolled has archived tumor tissue. Of the 191 patients enrolled in these trials, there are between 87 and 134 tumor samples that are suitable for bisulfite analysis. As part of the RTOG trial, all patients are required to submit tumor samples sufficient in size for analysis of MGMT promoter methylation by MS-PCR. As part of the RTOG trial, MS-PCR is performed by Oncomethylome (Durham, N.C.), and the MS-PCR data are compared with methylation sequencing data.

Tumor isolation and bisulfite treatment: Initial H&E sections are examined by pathologists to evaluate the extent of normal tissue versus tumor tissue in the sections. If a section contains greater than 90% tumor, then the unstained slides are scraped to obtain tumor tissue for DNA extraction. If a section has between 25% and 90% tumor, and the tumor and normal tissue are clearly demarcated, then the corresponding regions on the unstained slides containing tumor are scraped to isolate tumor tissues. If sections do not meet these criteria, then the block is sectioned further or other tissue blocks from the same patient are evaluated to identify suitable tumor sections. DNA from tumor scrapings is solubilized by incubation overnight at 50° C. in a proteinaseK/Tris/EDTA buffer. The reaction mixture is treated with bisulfite and DNA is subsequently desulfonated and purified using the EZ DNA methylation kit (Zymo Research, Orange, Calif.). Eliminating the initial DNA purification step prior to bisulfite modification significantly improved the DNA yield from this procedure such that sufficient DNA for eight PCR amplification reactions is routinely recovered from a single 10 micron thick section containing 1 cm² of tumor tissue. To complete both the MS-PCR and the MS-sequencing analyses, four PCR amplification reactions are performed.

Quantitative-MS-PCR: Methylation-specific PCR is performed on bisulfite-treated DNA using a multi-color fluorescent PCR reaction with a Taqman ABI 7900 system and ABI Genotyper software for analysis. Primers used for MGMT include the unmethylated forward primer 5′-FAM-TTT GTG TTT TGA TGT TTG TAG GTT TTT GT-3′ (SEQ ID NO:2) and the unmethylated reverse primer 5′-AAC TCC ACA CTC TTC CAA AAA CAA AAC A-3′ (SEQ ID NO:3) as well as methylated forward primer 5′-NED TTT CGA CGT TCG TAG GTT TTC GC-3′ (SEQ ID NO:4) and reverse primer 5′-GCA CTC TTC CGA AAA CGA AAC G-3′ (SEQ ID NO:5). PCR conditions are as follows: initial Taq-Gold activation for 10 minutes at 95° C., denaturation for 30 seconds at 94° C., annealing for 40 seconds at 59° C., and extension for 1 minute at 72° C. As controls, multiple concentrations of extracted DNA from two of the xenograft lines, GBM16 and GBM44, are processed in parallel with each run of clinical samples. Analysis of the thermocycler fluorescence data provide a quantitative measure of the relative number of methylated and non-methylated MGMT transcripts, and this ratio is used in the analysis of patient survival. The statistical analysis of the MS-PCR data is described below.

MGMT MS-sequencing: Methylation-specific DNA sequencing provides a comprehensive evaluation of methylation within the entire CpG island of the MGMT promoter (Matsukura et al., British Journal of Cancer, 88:521-529 (2003)). Specific methylation patterns within the CpG island may correlate directly with patient survival. The density of methylation within the CpG island also may be important for TMZ sensitivity. Using methylation specific DNA sequencing, the methylation status of multiple CpG sites within the CpG island is assessed and patterns of MGMT methylation are correlated with survival in three clinical trials. For methylation-specific sequencing of MGMT from paraffin-embedded tissue, bisulfite-treated DNA is used as a template in a non-methylation dependent amplification of the MGMT CpG island. The PCR products are cloned into a PCR cloning vector and the CpG island insert of 10 individual clones is sequenced by standard Sanger sequencing. In this way, each clone sequenced either contains a modified TpG (from an unmethylated CpG) or an unmodified CpG (from a methylated CpG site) at each CpG site, and the ratio of TpG to CpG sequences provides a graded measure of methylation from 0 to 100% at each CpG site. This graded measure of methylation is analyzed in relation to survival.

PCR cloning and sequencing: Bisulfite-modified DNA from each patient sample prepared for MS-PCR also is used as a template for PCR amplification in preparation for methylation specific sequencing. Since formalin-fixed DNA is more likely to be fragmented, the CpG island is amplified in two fragments using a nested PCR approach with the following primers and conditions. First PCR 5′ fragment: sense TTG GAI GGT ATI GTT ATT ATA GG (SEQ ID NO:6); antisense CCT AAA ACT CTA TAC CTT AAT TAA CC (SEQ ID NO:7); 40 cycles of 95° C./30 seconds, 50° C./30 seconds, 72° C./90 seconds. Second PCR 5′ fragment: sense TTA TTA TAG GTT TTG GAG GTT GTTT (SEQ ID NO:8); antisense CCT AAA ACT CTA TAC CTT AAT TAA CC (SEQ ID NO:9); 30 cycles of 95° C./30 seconds, 60° C./30 seconds, 72° C./60 seconds. First PCR 3′ fragment: sense GGT AAA TTA AGG TAT AGA GTT TTA GG (SEQ ID NO:10); antisense AAT AAA TAA AAA TCA AAA CIA CCC (SEQ ID NO:11); 40 cycles of 95° C./30 seconds, 50° C./30 seconds, 72° C./90 seconds. Second PCR 3′ fragment: sense GGT AAA TTA AGG TAT AGA GTT TTA GG (SEQ ID NO:12); antisense AAA AAT CAA AAC IAC CCC CC (SEQ ID NO:13); 30 cycles of 95° C./30 seconds, 60° C./30 seconds, 72° C./60 seconds. Both PCR products are cloned into the pCR2.1 TA cloning vector (Invitrogen). DH5α bacteria are transformed with the ligation reaction mix and plated on ampicillin-containing agar. A minimum of ten colonies are directly PCR amplified using vector-specific primers. The insert for each clone is sequenced in both directions. Samples are sequenced in the UCSF and Mayo Molecular Biology core facilities, and the DNA sequence data are analyzed using the Mutation Surveyor 3.01 software program (Softgenetics, PA). Automated sequence calling at each CpG site is confirmed by visual inspection of the DNA chromatogram-methylated CpG remains CpG, while unmethylated CpG is converted to TpG by bisulfite. The methylation status of each CpG site in each clone is recorded in an excel spreadsheet for use in statistical analyses.

Statistical analyses: The statistical analyses are based on a training dataset and a validation dataset. Samples from the NCCTG and UCSF clinical trials (estimated sample size of n=112 samples) are used to determine MGMT promoter methylation patterns associated with improved survival in newly diagnosed GBM patients treated with a TMZ-based therapy. Patient samples obtained from the RTOG clinical trial (n=200) are used to validate whether the identified patterns are associated with improved survival.

A preliminary analysis of the training set (samples from the NCCTG and UCSF trials) is done to determine the type of association between methylation status of a CpG site and survival. Three possible association types are dose-dependent, step, or threshold. A dose-dependent effect suggests that methylation status can be treated as a continuous variable. A step effect suggests that methylation data can be divided into discrete increments for an analysis of associations between incremental changes in methylation and survival. A threshold effect suggests there is a single cutpoint for methylation values such that on one side there is no effect and on the other side there is a complete effect. The objective of the preliminary analysis is to determine the best form (i.e. coding) of the methylation status for each site: continuous (dose-dependent), ordinal (step function), or binary (threshold relationship).

The MGMT MS sequencing data yield a value of 0 to 10 for each of the 98 CpG sites corresponding to the number of clones out of 10 that are methylated. In the statistical analysis, this variable can be treated as continuous (using the 0 to 10 scale), as ordinal if each site is categorized into three classes (0—unmethylated, 1—partially methylated, or 2—fully methylated), or as a dichotomous variable (methylated versus not methylated) depending on whether or not the number of methylated clones are above a specified threshold. To determine the best form/coding of the variable to use, the relationship between each variable and survival is assessed. Rpart (Therneau and Atkinson, An Introduction to Recursive Partitioning Using the RPART Routines, Department of Health Sciences Research, Section of Biostatistics, Technical Report #61, Mayo Clinic, Rochester, Minn., USA, (1997)) is used with survival as the outcome and each site as a variable (98 rpart models, one for each site) to determine the number of cutpoints (one, two, or more) for a site. Next, univariable Cox models are applied with the three different coding methods for the variable: continuous, ordinal, and binary (cutpoints for ordinal and binary coding for the variable are determined from rpart). From these analyses, it is determined whether there appears to be a dose-dependent effect in the association between methylation status at a site, a step effect, or a threshold effect, depending on which Cox model appears to fit the data best. A similar univariable analysis is performed for the most appropriate coding of quantitative MS-PCR as an explanatory variable. Note that this variable is continuous with a range from 0 to 1 but can also be coded as ordinal or binary.

After the best form/coding for the methylation status at each site is determined, three different Cox proportional hazards models are generated and their predictive capabilities are compared. Each of the models uses survival as the outcome variable and differs only in the explanatory variables used. The different models are as follows.

Model 1: explanatory variable is the amount of methylation across the 98 sites. If methylation status at each site is treated as a continuous variable, the amount of methylation is the sum of 98 variables with each variable ranging from 0 to 10. If methylation status at a site is treated as binary (ordinal), then the amount of methylation is the sum of 98 variables, with each variable being either a 0 or 1 (0, 1 or 2), and the sum again is treated as continuous.

Model 2: explanatory variables are the sites identified by random forests as being important methylation sites relative to survival. The Random Forests tool is an expansion of the tree concept in rpart (Breiman, Machine Learning, 45:5-32 (2001)). Instead of just growing one tree, thousands of trees are grown and then averaged together. Briefly the algorithm grows each tree on an independent bootstrap sample of the data. At each node, m sites (variables) are randomly selected out of the 98 CpG sites and the best split is found based on the m variables. The tree is grown to maximum depth. This process is repeated 5000 times and the trees are averaged to get predictions. The Random Forest tool is useful for sorting through a large number of variables and coming up with a list of the top 5 or 10 to investigate further. The purpose of this model is to determine the combination of CpG sites (or pattern of methylation) that yields the best prediction. The sites are the potential explanatory variable and survival is the outcome variable (with censored values). Random forests yield sites that appear associated with survival and the pattern (or combination) of these important methylation sites are determined using rpart. Specifically, rpart is performed using only the sites selected as important by random forests. A multivariable Cox proportional hazard model is created with the explanatory variables being the sites identified by the random forest/rpart analysis (including any identified interactions).

Model 3: explanatory variables are the MS-PCR result. The form of the explanatory variable, MS-PCR, is continuous (range of 0 to 1), ordinal, or binary, depending on the results of the preliminary analysis described above. The outcome variable is survival and a Cox proportional hazard model is used.

To determine which of the three models above yields the best predictor, the performance of the models is compared using an enhanced bootstrap approach (Harrell, Regression modeling strategies: with application to linear models, logistic regression, and survival analysis. New York: Springer-Verlag, 2001). Instead of estimating an accuracy index directly from the original sample, the enhanced bootstrap uses a slightly more indirect approach by estimating the bias due to over-fitting. This is called the optimism value. After the optimism is estimated, it is subtracted from the accuracy derived from the original sample to obtain a bias-corrected estimate of predictive accuracy. This approach allows the predictive accuracy of models to be compared with different numbers of variables. The model with the best (bias-corrected) estimate of accuracy is declared the best.

For the training analysis, there are 66 patients enrolled in the UCSF study and 138 patients enrolled in the NCCTG study, for a total of 204 patients. Tissue specimens are obtained from about 80% of enrolled patients, and about 70% of collected tissue specimens have a suitable amount of material for the analyses. Results similar to the EORTC study, in which 46% of patients with methylated MGMT tumors compared to 14% for non-methylated tumors were alive at 2 years (Hegi et al., N Engl J Med, 352:997-1003 (2005)), and assuming a 2-tailed log rank test with an alpha of 0.05, 112 samples (200×0.8×0.7) have 98% power to detect a difference in survival at least as large as that seen in the EORTC study.

Although mechanisms to avoid overfitting are employed in the training set for Model 2 (use of random forests and rpart, which have internal cross-validation) and an enhanced bootstrap is used to get bias-corrected estimates of predictive accuracy of the models, the predicted accuracy of the models may be biased and may not generalize. To gain further evidence that the model deemed in the training phase to be the best remains the best in an independent dataset, the three models developed in the training phase are applied to the independent validation dataset of n=200 RTOG samples. The predictive accuracy (or performance) of these models is evaluated using the enhanced bootstrap described above. Model 2 can be superior in the training dataset and in the validation dataset. With 200 samples and assuming a 2-tailed log rank test with an alpha of 0.05, a difference in survival is detected that is at least as large as that seen in the EORTC study; i.e., there is 100% power to detect a difference as large as that observed in the EORTC trial, with any of the models. If the results from the training phase are not consistent with those obtained in the validation phase, i.e., different models are determined to be the best in terms of predictive accuracy in the two phases, then all the data (n=312) are pooled and the three models are developed and evaluated as described above for the training phase.

Additional approaches: There are several approaches for evaluating promoter methylation. EpiTYPER (Sequenom, San Diego, Calif.) provides quantitative methylation analysis using mass spectrometry. After bisulfite modification and PCR amplification, reverse-strand in vitro RNA transcription is performed. The resulting RNA product is cleaved by RNaseA at U or C and the resulting fragments are evaluated by MALDI-TOF mass spectrometry (Ehrich et al., Proceedings of the National Academy of Sciences of the United States of America, 102:15785-15790 (2005)). Analysis of the cleavage products produces a unique signal pair pattern for methylated and non-methylated templates, and the relative abundance of these products is calculated to provide a quantitative measure of methylation across the amplified template region.

In the MethylScreen technique (Orion Genomics, Saint Louis, Mo.), cleavage of genomic DNA with methylation-specific restriction enzymes is followed by real-time multiplex quantitative PCR to define the extent of cleavage at specific CpG sites. By eliminating the use of bisulfite, this technique may be useful for evaluating smaller biopsy specimens.

Other factors that may influence sensitivity to TMZ are examined in conjunction with MGMT status. p53 status is examined in the NO177 specimens, although p53 status did not correlate with TMZ sensitivity in the xenograft panel or in other cell and xenograft models (Leuraud et al., Cancer Res, 64:4648-4653 (2004), Roos et al., Oncogene, 23:359-367 (2004), D′Atri et al., Molecular Pharmacology, 54:334-341 (1998); Xu et al., Journal of Neuro-Oncology, 74:141-149 (2005); Hirose et al., Cancer Res, 61:1957-1963 (2001), Middlemas et al., Clinical Cancer Research, 6:998-1007 (2000), Bocangel et al., Clinical Cancer Research., 8:2725-2734 (2002)). The integrity of mis-match repair (MMR) also is evaluated in the patient samples (Thibodeau et al., Cancer Research, 56:4836-4840 (1996); Thibodeau et al., Cancer Research, 58(8):1713-1718 (1998)). Integrity of MMR can be important for TMZ sensitivity in colon cancer, but microsatellite instability (MSI), which is indicative of defective MMR, is seen in only 5% of GBM patients (Martinez et al., Oncology, 66:395-403 (2004); Martinez et al., Journal of Cancer Research & Clinical Oncology, 131:87-93 (2005)). Components of the NF-kappaB pathway may be important modulators of TMZ sensitivity in GBM tumors (Bredel et al., Journal of Clinical Oncology, 24:274-287 (2006)). Although this may reflect the influence of NF-kappaB signaling on MGMT regulation, the nuclear translocation of NF-kappaB, which is indicative of activation, is evaluated by IHC (Ismail et al., Prostate, 58:308-313 (2004); Santoro et al., Journal of Pathology, 201:466-472 (2003)).

Additional experiments: An assay is developed and validated that is more clinically applicable than the bisulfite-sequencing technique. A real-time multiplex MS-PCR assay is used to evaluate the methylation status of multiple sites across the CpG island. Methods such as EpiTYPER or MethylScreen are validated on clinical and xenograft GBM specimens.

Delineating MGMT Methylation Patterns Associated with TMZ Sensitivity in the GBM Xenograft Model

The following three complimentary approaches are used to determine whether methylation at specific CpG sites has a significant influence on MGMT expression and TMZ responsiveness.

(1) Extended flank tumor passage can result in increased MGMT promoter methylation (FIG. 6) and an associated decrease in TMZ resistance (Table 2). An extensive tumor archive of xenograft specimens is available from each tumor generation, and changes in MGMT promoter methylation are directly compared with changes in MGMT polypeptide and mRNA expression across multiple generations in 17 GBM xenograft lines. Through a comprehensive statistical analysis of these data, specific CpG methylation sites are identified that are important regulators of MGMT expression across multiple tumor lines. The term “generation,” as used in the section of this document under the sub-heading “Delineating MGMT methylation patterns associated with TMZ sensitivity in the GBM xenograft model,” is used to describe the number of serial passages of any xenograft line since the initial implantation of the patient tumor sample. For example, generation 1 refers to the original tumor xenograft established from the patient sample, generation 2 refers to those tumors that were serially transplanted from the generation 1 xenograft, and generation 3 refers to those tumors derived from second generation tumors.

(2) TMZ responsiveness has been assessed in multiple xenograft generations for six xenograft lines. In conjunction with additional sensitivity testing, methylation changes in specific CpG sites that are associated with increased TMZ sensitivity are identified. Both the first and second approaches provide an independent validation of the critical CpG methylation sites and/or regions associated with prolonged survival with TMZ therapy that are identified as described above.

(3) An MGMT-promoter luciferase assay is used to interrogate the influence of specific CpG methylation sites on MGMT promoter activity.

Evaluating CpG methylation changes within the MGMT promoter: Specific CpG methylation sites important for regulating MGMT mRNA and polypeptide levels are identified by evaluating multiple xenograft generations in 17 xenograft lines. The primary patient tumor tissue sample used to establish each xenograft line and flank tumor tissue from each generation of each xenograft line are available in tissue archives, and these samples are used to analyze changes in MGMT methylation over time. For each of the 17 xenograft lines, MS-PCR and methylation-specific sequencing of the MGMT promoter are performed in the original patient tumor sample (G0), the first generation flank tumor (G1) derived directly from patient tumor implantation, and samples from G2, G5, G10, and G15. Some lines have been passaged to 40+ generations, and in these xenograft lines additional samples from G20, G30, and G40 also are processed. These results are correlated with quantitative immunoblotting analyses of MGMT polypeptide levels and quantitative RT-PCR analyses of MGMT mRNA levels. Based on preliminary data, extended flank tumor propagation can be associated with reduced MGMT expression that is linked to discrete changes in MGMT methylation patterns. Delineating these associations can help identify specific CpG methylation sites that are important regulators of MGMT expression. Because methylation within the promoter occurs in regions associated with a closed chromatin structure, these data may indicate specific regions within the MGMT promoter with key roles in transcriptional regulation.

Archived flash-frozen flank tumor samples and the original frozen patient tumor specimens, which are all stored at −80° C., are analyzed. With the exception of the original patient tumor sample, three specimens from each of the indicated tumor generations are analyzed. For each analysis, specimens are ground using a mortar and pestle under liquid nitrogen, and the resulting tumor powder is used for the protein, mRNA, and DNA analyses. The MS-PCR assay described herein is used, as is the DNA sequencing method described herein, except that the entire CpG island can be amplified from bisulfite-treated DNA as a single fragment, since DNA derived from frozen samples is in optimal condition. Also, the PCR amplification products derived from the bisulfite-modified DNA are used directly for sequencing instead of being subcloned into a TA vector. Through direct inspection of the chromatograms, methylation at each CpG site is classified as unmethylated, hemi-methylated, or methylated and assigned a score of 0, 1, or 2, respectively. The methylation status at each site within a generation is described as an average of this score across all three xenograft samples. Quantitative RT-PCR is performed as described elsewhere (Tanaka et al., Cellular & Molecular Neurobiology, 25:1067-1071 (2005); Tanaka et al., International Journal of Cancer, 103:67-72 (2003)). Briefly, following mRNA isolation, cDNA is prepared in a reaction using reverse-transcriptase. cDNA then is used in a quantitative PCR amplification using primers specific for MGMT (5′-CCTGGCTGAATGCCTATTTC (SEQ ID NO:14), and 5′-GATGAGGATGGGGACAGGATT (SEQ ID NO:15)) and primers for human f32-microglobulin as an internal control (5′-TTCTGGCCTGGAGGGCATCC (SEQ ID NO:16) and 5′-ATCTTCAAACCTCC-ATGATG (SEQ ID NO:17)). Cycle parameters are as follows: initial melting at 95° C. for 10 minutes followed by 50 cycles at 95° C. for 30 seconds, 60° C. for 40 seconds, and 72° C. for 30 seconds using a Taqman ABI 7900 system real-time PCR system. To allow quantitation of copy number, the MGMT and J32-microglobulin PCR products are cloned into the pCR 2.1 TA-cloning vector, and serial dilutions of purified plasmid for each product are run simultaneously with each amplification run. Expression levels of MGMT polypeptide are evaluated by Western blotting of each tumor sample as was done for FIG. 1. Graded amounts of recombinant MGMT protein are loaded on each gel to facilitate quantitative assessment of MGMT expression levels relative to β-actin by film densitometry.

The statistical analyses focus on two questions. First, it is analyzed whether the subset of CpG sites, identified as being predictive of TMZ response as described above, are also associated with regulation of MGMT expression or TMZ responsiveness in the xenograft model. The MS-PCR data and the CpG methylation data for the corresponding nine CpG sites also are analyzed with a similar intent of corroborating the results obtained as described above. Second, methylation changes at all 98 CpG sites are analyzed in order to identify other important CpG methylation sites that modulate MGMT expression and/or TMZ sensitivity. Initially the data are explored graphically. For each xenograft line, the methylation status of each site within a generation is described as an average across all three xenograft samples. Similarly, within each xenograft line, the polypeptide expression level at each site within a generation is summarized as an average of the polypeptide expression level across all three xenograft samples. Some examples of plots that are generated include the following. A smoothed dotplot of methylation score (averaged across all CpG sites) versus generation for each xenograft line is used to explore whether overall methylation increases over generation passage and whether the pattern is similar across all xenograft lines. A smoothed dotplot of methylation score (averaged across all xenograft lines) versus CpG site by generation is used to identify potentially important CpG sites within a generation and across all generations. A smoothed plot of methylation score (averaged across all generations and xenograft lines) versus CpG site also is generated.

A simple univariable analysis is done using a two-sample t-test to evaluate the difference in methylation between generations by MS-PCR or at any specific CpG site by sequencing. A more general analysis is performed using a linear mixed regression model with methylation status at each site as an outcome variable (one model per CpG site). These analyses use the observed methylation status for each mouse at each site (rather than averaging over the three mice within a xenograft line). In this model, xenograft line and passage generation are fixed effects and the mouse within a xenograft line is treated as a random effect with correlation proportional to the passage. From this model, a trend test for passage effect can be used to explore whether there is a passage generation effect on methylation status. To determine whether there are sites whose change over time is correlated, linear regression is performed between each of the sites identified as changing over time. Specifically, the x and y variables are the time of the change for site 1 and 2, respectively. The correlation coefficient is estimated.

An analysis also is performed using a linear mixed regression model with polypeptide or mRNA expression level as the outcome. In this model, xenograft line and passage are treated as fixed effects. Mouse within each xenograft line is treated as a random effect with auto-regressive (AR) correlation structure. This model helps to understand passage generation effect on polypeptide or mRNA expression levels. To explore the relationship of polypeptide or mRNA expression level and methylation status, sites identified as being important are used in a linear mixed model with polypeptide or mRNA expression levels as the outcome variable and CpG sites, xenograft line, and passage generation as fixed effects and mouse within each xenograft line as a random effect. This allows the association between CpG sites and polypeptide or mRNA levels to be determined. For this analysis, each CpG site is assessed individually (adjusting for xenograft line, passage generation, and mouse) as well as a group (all sites in the model simultaneously). As a secondary analysis, the association with each remaining site is explored while adjusting for xenograft line (fixed) passage generation (fixed) and mouse (random). Collectively, these analyses establish a model for understanding how specific changes in CpG methylation influence MGMT mRNA and MGMT polypeptide expression levels.

Performing in vivo TMZ sensitivity analysis in early and late generation xenografts: If MGMT promoter methylation is an important determinant of TMZ responsiveness in GBM tumors, then evidence of progressive MGMT promoter methylation may be associated with enhanced TMZ responsiveness. To test this idea and to identify specific methylation sites associated with TMZ sensitivity, changes in MGMT promoter methylation patterns are compared with changes in TMZ sensitivity in multiple generations within xenograft lines. As detailed in Table 2, TMZ response has been determined in an orthotopic xenograft survival assay for at least two generations in each of six xenograft lines, and in three of the five non-methylated MGMT tumors (GBM10, 14, and 44 by MS-PCR). The efficacy of TMZ therapy was significantly increased in later generations, despite being treated with nearly half the dose of TMZ (66 mg/kg×5 days) as compared to the initial early generation studies (120 mg/kg×5 days). Archived tumor samples from these experiments are analyzed, and the analysis is expanded by completing additional TMZ therapy evaluations in late tumor generations (at least 10 generations removed from the previous TMZ sensitivity determination) for each of the six tumor lines listed in Table 2. In addition, TMZ response is tested in early passage xenograft lines restored from cryopreserved tumor specimens using the TMZ dosing regimen of 66 mg/kg/day×5 days. In this way, TMZ sensitivity is determined for all tumor lines in early passage lines (generation 5 or less), and at least two determinations are made in later generations. Within any one tumor line, specific CpG sites or CpG methylation patterns are identified that are associated with changes in TMZ sensitivity in essentially the same genetic background.

TMZ responsiveness is evaluated in an intracranial therapy model as was done for the studies reported in Tables 1 and 2. As described above, TMZ sensitivity is evaluated in late-generation and early-generation xenografts from six lines (GBM6, 10, 12, 14, 43, and 44). For each experiment, short-term cell cultures established from the relevant flank tumor xenografts are implanted intracranially using a stereotactic jig. Two weeks prior to the expected onset of symptoms suggestive of significant tumor burden, mice are randomized into two groups of 10 mice each and dosed by oral gavage with vehicle control or TMZ (66 mg/kg) for 5 days. Mice are followed until moribund and after euthanasia, brains are removed and sectioned along the old injection tract. The front half is embedded in OCT and frozen, while the opposing half is fixed in formalin and paraffin embedded. Survival is summarized using Kaplan-Meier estimates. Within any given tumor line, the change in sensitivity with generation time is analyzed by a Cox proportional hazard model using the following covariates: treatment (TMZ vs. placebo), generation, and treatment by generation interaction. The interaction term can be used to determine whether the survival difference between different generations is significant.

Brain tumor specimens from all moribund mice treated in therapy trials are routinely archived and are available for MGMT methylation analysis. Intracranial tumor specimens from placebo-treated mice from each therapy experiment are used for an analysis of MGMT promoter methylation sequencing. This allows for a robust correlation between changes in MGMT methylation status and TMZ responsiveness across the specific tumor generations that are evaluated. Methylation sequencing is performed on intracranial tumor tissue from six placebo treated mice from each therapy experiment. Using the OCT-embedded tumor tissues, five 10 micron brain cross-sections are cut and tumor is delineated on an H&E stained section by a board certified neuropathologist with experience in the analysis of intracranial xenograft tumor in nude mice. The H&E section is superimposed with unstained slides to allow removal of tumor tissue for processing and direct methylation-specific sequencing as described above. The methylation status of each CpG site is scored as 0 (unmethylated), 1 (hemi-methylated), or 2 (fully methylated), and significant differences in methylation status between experiments at any one CpG site are determined by performing an analysis similar to that described above. A linear mixed model is used to assess whether there are significant differences in methylation status between experiments at any one CpG site. The outcome variable is methylation status and the CpG site, xenograft line, and passage generation are fixed effects. Mice within a xenograft line are a random effect. All sites found to have changed significantly are explanatory variables in a linear model to determine the association of these changes with TMZ responsiveness. Linear regression is used with TMZ responsiveness (calculated as the ratio of the median survival of the TMZ treated group to the median survival of the placebo group) as the outcome variable, and CpG site methylation status, passage generation, and a CpG site/passage generation interaction as explanatory variables. It is determined whether there is a significant interaction between the methylation status at a site and generation in relationship to TMZ sensitivity.

Restoration of MGMT expression in late GBM xenograft lines: The contribution of progressive MGMT promoter methylation to increased TMZ sensitivity of late generation tumors identified in the previous section is tested using two approaches. First, an MGMT-encoding lenitvirus is used to re-express MGMT in late generation tumor lines to test whether restoration of MGMT expression reverses the acquired TMZ sensitivity. Second, late-generation xenograft lines are treated with a DNA methyltransferase (DNMT) inhibitor to test whether loss of methylation induces re-expression of MGMT and increased TMZ resistance. Since reduced MGMT methylation or constitutive expression of exogenous MGMT can have similar effects on TMZ responsiveness both in vitro and in vivo, these studies are performed in cell culture. Short-term cell cultures that are used to establish any intracranial therapy experiment (e.g., those shown in Table 2) are routinely cryopreserved, and this resource is used to re-establish short-term cultures for the experiments described in this section.

High-level MGMT expression is restored in late generation xenograft lines using an HIV-based lentiviral transduction system. Short-term xenograft cultures are routinely transduced using either the pHR-SIN-Luc luciferase construct or the pHR-SIN-DsRed red fluorescent protein construct, and greater than 95% transduction efficiency is routinely achieved with resulting high-level luciferase or DsRed expression, respectively. An MGMT-expressing pHR-SIN-MGMT construct is created by swapping an MGMT expression cassette for the DsRed expression cassette, and the pHR-SIN-DsRed vector is used as a vector control. Early and late generation short-term cultures are transduced with either of the two vectors. After 96 hours, MGMT expression levels are compared between transduced and non-transduced cell lines by Western blotting, and transduction efficiency is estimated by fluorescence microscopy. MGMT transduction is considered to be successful if MGMT expression levels following transduction are similar or greater than endogenous MGMT levels in early generation tumor lines, and if transduction efficiency is greater than 90%. Successfully transduced lines are evaluated for TMZ sensitivity. In addition, a bi-cistronic expression vector encoding both DsRed and firefly luciferase has been developed, and the luciferase expression cassette can be replaced by MGMT to produce a vector that allows direct visualization of transduction efficiency with an MGMT-encoding vector.

TMZ sensitivity is assessed in a 96 well plate methylene blue assay. This assay relies on staining of cellular polypeptides and has been used extensively in the laboratory for analysis of TMZ sensitivity. For all assays, unmodified early and late generation cell cultures are used in parallel with transduced cell lines. For each sub-line, cells are plated at 5000 cells per well in quadruplicate wells of 96 well plates and treated with graded concentrations of TMZ up to 100 μM. After incubation for 96 hours, cells are fixed, stained with methylene blue, and absorbance of each well is measured in a spectrophotometer. Each experiment is performed three times and the relative change in absorbance is pooled across experiments for analysis. Differences in TMZ sensitivity between the manipulated and original cell cultures tested from each xenograft line are compared using a dose response analysis. A general linear model is used with cell survival rate as the outcome variable. Slopes of lines are compared to test for group differences (e.g., early generation versus late generation+MGMT restoration, late generation alone versus late generation+MGMT restoration). When changes in TMZ sensitivity associated with tumor passage are solely due to suppression of MGMT expression levels, then re-expression of MGMT in the late passage lines can restore TMZ resistance to the same level as non-transduced early generation xenograft lines.

The effects of MGMT methylation on MGMT expression and TMZ sensitivity also are tested using the DNMT inhibitor 5-azacytidine. Short-term cultures from both early and late generation tumors are treated with 1 μM 5-azacytidine for 7 days (Danam et al., Mol Cancer Ther, 4:61-69 (2005), Pieper et al., Cancer Communications, 3:241-253)1991)). Effects on MGMT expression are evaluated by Western blotting and effects on MGMT methylation are evaluated by bisulfite sequencing. The influence on TMZ sensitivity is evaluated in a methylene blue assay, as described above. When progressive MGMT methylation leads to suppression of MGMT expression and increased TMZ sensitivity in late generation xenografts, then reversal of DNA methylation by inhibition of DMNT activity can restore MGMT expression levels to those seen in early generation tumors in those tumor lines that originally had high-level MGMT expression.

The purpose of these experiments is to confirm that suppression of MGMT accounts for progressive changes in TMZ sensitivity. When MGMT re-expression does not completely restore TMZ resistance in late-passage xenograft lines, then the integrity of other molecular pathways involved in processing of TMZ damage is evaluated. For example, base-excision repair pathways and DNA damage checkpoint pathways are evaluated (Hirose et al., Cancer Research, 61:5843-5849 (2001); Silber et al., Clin Cancer Res, 8:3008-3018 (2002); Liu et al., Clinical Cancer Research, 5:2908-2917 (1999)). These DNA damage processing pathways are investigated by Western blotting for key components within these pathways (e.g., DNA-PK, DNA ligase IV, Ku70/80, ATM, Rad51, Chk1, Chk2, for DNA dsb processing and repair, and PARP, AP endonuclease, DNA glycosylases, DNA polymerase beta, XRCC1, and DNA ligase I or III for BER). When there are defects in DNA damage processing, then cross-sensitivity to other classes of DNA damaging agents can occur in late-generation tumors. For example, defects in DNA double-strand break processing can lead to hypersensitivity to ionizing radiation, and defects in BER can lead to sensitivity to methylating agents such as methyl methanesulfonate (MMS), the efficacy of which is not affected by MGMT activity (Bobola et al., Clinical Cancer Research, 2:735-741 (1996), Schwartz et al., Carcinogenesis, 10:681-685 (1989)). Comparison of gene expression profiles between early and late passage xenografts are performed to identify alterations in expression of potential candidate repair and survival pathways involved in TMZ responsiveness.

Validating key CpG methylation sites within the MGMT promoter: The influence of key CpG methylation sites identified as described above are validated using an MGMT promoter luciferase construct. Promoter regulatory elements are commonly evaluated using luciferase-promoter constructs, where a promoter of interest is used to drive expression of a luciferase reporter polypeptide. By comparing the luciferase expression with a wild-type promoter versus truncated or mutated promoters, the importance of specific regions or regulator sites is evaluated. This technique has been used to evaluate MGMT promoter regulation or promoter inducibility following DNA damage (Nakagawachi et al., Oncogene, 22:8835-8844 (2003), Grombacher et al., Carcinogenesis, 17:2329-2336 (1996); Bhakat and Mitra, Carcinogenesis, 24:1337-1345, (2003); Biswas et al., Oncogene, 18:525-532 (1999); Sato et al., Oncology Reports, 13:899-906 (2005)). Using a similar approach, in vitro methylation of the MGMT promoter by restriction methylases has been used to evaluate the influence of MGMT CpG island methylation on promoter activity (Nakagawachi et al., Oncogene, 22:8835-8844 (2003), Bhakat and Mitra, Carcinogenesis, 24:1337-1345 (2003)). To avoid the confounding effects of methylation elsewhere in the reporter construct, in both studies, the promoter region was methylated in vitro, ligated into an unmethylated luciferase expression vector, and the purified ligation product was used directly for transfection into cells. A similar approach is used to compare the effects of in vitro methylation of a wild-type MGMT promoter versus a modified promoter in which key CpG sites are mutated singly or in combination. The same technique is used to validate the importance of CpG methylation at specific sites within the MGMT promoter that are associated with prolonged survival following TMZ-based therapy and/or suppression of MGMT expression. When methylation at the specific CpG sites is critical for silencing MGMT expression, then mutation of these sites can prevent suppression of MGMT promoter activity in comparison to the wild-type promoter.

An MGMT promoter-luciferase reporter construct described elsewhere (Bhakat and Mitra, Carcinogenesis, 24:1337-1345, 2003) was obtained, and the promoter construct was observed to have high luciferase activity when transduced into U87 and U251 cells. A mutant MGMT promoter is created in which one or more key CpG methylation sites are mutated from CpG to ApG. This is accomplished using sequential site-directed mutagenesis (QuikChange Site Directed Mutagenesis Kit, Stratagene). The wild-type and mutant plasmids are subjected to a double digest with Kpn I and Nhe I, and the promoter region from each plasmid is isolated. Promoter DNA is subjected to in vitro methylation. Briefly, plasmids are incubated with SssI (CpG) methylase in the presence (methylated) or absence (mock-methylated) of S-adenosylmethionine. Methylation reactions are monitored by methylation-sensitive restriction endonucleases. Following methylation, the promoter constructs are re-ligated into the original vectors. After purification with a QIAquick Gel Extraction Kit (Qiagen), the vectors are used in a transient transfection of U87 cells using Fugene6. As a transfection control, cells are co-transfected with a constitutively expressing Renilla Luciferase construct. After incubation for 24 hours, cells are harvested, and luciferase activities are measured using a Dual-Luciferase Reporter Assay System (Promega).

Evaluating changes in CpG island methylation in other genes: To determine whether the observed evolution of MGMT promoter hypermethylation with increasing generation reflects a more global process of epigenetic silencing of genes, and whether this process contributes to tumor progression, changes in CpG methylation within genes epigenetically regulated in GBM are evaluated (Gonzalez-Gomez et al., International Journal of Oncology, 22:601-608 (2003); Ohta et al., Oncology Reports, 16:957-963 (2006); Nakamura et al., Brain Pathology, 11:159-168 (2001); Nakamura et al., Laboratory Investigation, 81:77-82 (2001); Park et al., Journal of Korean Medical Science, 15:555-559 (2000)). Bisulfite-treated DNA samples prepared from the primary tumor (G0), and early (G2, G5, G10) and late (G20 and G30) xenograft generations are evaluated for changes in CpG methylation by MS-PCR in the following cell cycle-regulatory genes: p14ARF, p15INK4b, p16INK4A, RB1, TP53, p′73, p21Waf1/Cip1 and p27Kip1. Details of the MS-PCR reactions with regards to PCR primers and conditions are described elsewhere (Gonzalez-Gomez et al., International Journal of Oncology, 22:601-608 (2003); Ohta et al., Oncology Reports, 16:957-963 (2006)). Epigenetic modifications in cell cycle regulatory genes are the focus based on the observation that tumor growth rates significantly increase in late generation tumors. Silencing of one or more CDK-inhibitory genes (p14, p15, p16, p21, and/or p27) by progressive methylation can occur with increasing generation.

### Evaluating the Effects of TMZ Therapy on MGMT Methylation Patterns

Emergence of TMZ resistance in GBM is a major clinical problem, and understanding the mechanisms of this phenomenon is important for developing more effective therapies. Intra-tumoral cellular heterogeneity of MGMT polypeptide expression in patient samples has been described (Friedman et al., Journal of Clinical Oncology, 16:3851-3857 (1998); Rood et al., Neuro-Oncology, 6:200-207 (2004), Lee et al., Carcinogenesis, 17:637-641 (1996)). Given the influence of MGMT on TMZ responsiveness, this heterogeneity suggests that TMZ-sensitive and TMZ-resistant sub-populations of cells may co-exist within tumors. When this is true, then TMZ therapy can result in progressive enrichment of TMZ resistant cells and lead to diminishing efficacy of TMZ with subsequent treatments. Consistent with this idea, an in vivo study with GBM14 demonstrated that a single dose of TMZ resulted in a 56 day prolongation in survival relative to placebo, while a second dose of TMZ delivered at the time of tumor recurrence added only an additional 22 days of survival (FIG. 7). Studies described elsewhere have demonstrated heterogeneity in genomic methylation patterns within a tumor cell population, and heterogeneity in methylation patterns within the MGMT promoter (Yamada et al., Journal of Biological Chemistry, 276:19052-19058, (2001); Danam et al., International Journal of Oncology, 18:1187-1193 (2001)). Reduced efficacy of the second TMZ cycle in the in vivo study may have been due to selection of pre-existing tumor cells with MGMT methylation patterns that confer high-level MGMT expression and TMZ resistance. In a separate study with GBM12, it was observed that discrete changes in the methylation of multiple CpG sites within the MGMT promoter were associated with the development of TMZ resistance (FIG. 8). TMZ resistance may develop in GBM due to inactivation of mismatch repair proteins (Hunter et al., Cancer Research, 66:3987-3991 (2006); Friedman et al., Cancer Res, 57:2933-2936 (1997)). The xenograft model is used to evaluate the extent of MGMT promoter methylation changes after TMZ therapy and to determine whether these changes result in TMZ resistance.

Deep amplicon sequencing is used to determine whether cells with genetic alterations, e.g., altered MGMT methylation patterns or mutations in MMR or other genes, can be detected at low frequency in TMZ-naïve tumors. In conjunction with an improved understanding of TMZ resistance-induction mechanisms, such an approach can used as a more robust predictor of TMZ responsiveness in patients, and potentially can identify a priori the anticipated mechanism of TMZ resistance. A series of 5 experiments is performed to: (1) evaluate the prevalence of MGMT methylation pattern changes following a single cycle of TMZ in 17 xenograft lines, (2) evaluate the development of intrinsic tumor cell resistance to TMZ during multiple cycles of TMZ therapy in 7 xenograft lines, (3) evaluate relationships between changes in MGMT expression levels, MGMT promoter methylation patterns, and development of TMZ resistance in association with (2), (4) evaluate whether defects in MMR contribute to the development of TMZ resistance in (2), and (5) evaluate whether TMZ resistance develops by selection of pre-existing TMZ resistant clones using deep amplicon sequencing.

Evaluating changes in MGMT methylation patterns following TMZ therapy: Preliminary data demonstrate relatively rapid development of TMZ resistance in association with changes in MGMT methylation patterns, and this change in MGMT methylation may result from selection of pre-existing tumor clones with MGMT promoter methylation patterns which confer TMZ resistance upon the cell. To obtain a better understanding of how prevalent this selection phenomenon might be, MGMT promoter methylation sequencing is performed on tumor samples from 17 GBM xenograft lines following a single cycle of TMZ. As detailed in Table 1, the efficacy of TMZ has been tested in these xenograft lines, and the brains from all moribund mice on these experiments were removed, sectioned, and frozen in OCT for use in additional studies. The intracranial tumor burden is significant in moribund mice, and these tumor samples provide ample tissue for analysis. With these archived tumor samples, the methylation status of each CpG site is compared between placebo and TMZ-treated mice using bisulfite sequencing in each xenograft line to understand how commonly MGMT promoter methylation patterns change following TMZ therapy and whether there are specific methylation pattern changes that are shared across multiple tumor lines. Moreover, the extent of change in methylation patterns is correlated with the median survival prolongation with TMZ therapy in each tumor line. When the extent of survival prolongation in the orthotopic survival studies reflects the extent of cell killing, and when changing MGMT methylation patterns reflect the loss of sensitive tumor cells and regrowth of resistant tumor clones, then the greatest change in methylation patterns can occur in the most TMZ sensitive tumors, while minimal changes in methylation patterns can occur in the most resistant xenograft lines.

For each tumor line, mouse brain samples from moribund mice are sectioned and a representative section from each tumor is stained by H&E. Using the H&E as a guide, gross tumor is scrapped from serial sections and processed for bisulfite sequencing as described above. Tumor samples from 6 placebo and 6 TMZ-treated mice are analyzed for each xenograft line, and as described above, methylation at each CpG site is scored from 0 to 2. Changes in methylation status at each CpG site are assessed with a linear mixed model. The outcome variable is the methylation status at a CpG site and the explanatory variables are xenograft line (fixed), treatment (fixed), and mouse (random). It is determined whether there is an association between the treatment and CpG site methylation status.

Evaluating development of TMZ resistance during cyclical TMZ therapy: Preliminary data demonstrate reduced efficacy of a second TMZ treatment in an orthotopic survival study using GBM14 (FIG. 7), and this may reflect the emergence of TMZ resistance. It is determined whether this is a common phenomenon by testing the efficacy of one or more cycles of TMZ in seven xenograft lines of varying TMZ sensitivities. The intrinsic TMZ sensitivity of tumor cells recovered from moribund mice treated with placebo or TMZ is evaluated in an ex vivo survival assay. On the basis of TMZ sensitivity determinations (Table 1), seven xenograft lines exhibiting a range of TMZ sensitivities and MGMT methylation patterns were selected to be studied. Of the non-methylated MGMT tumors (by MS-PCR), GBM14 (558% survival prolongation), GBM34 (294% survival prolongation), and GBM43 (87% survival prolongation) are tested. Of the methylated MGMT tumors, GBM8 (110% survival prolongation), GBM12 (253% survival prolongation), GBM39 (>235% survival prolongation), and GBM16 (579% survival prolongation) are tested.

For each xenograft line, short-term cell cultures derived from early generation flank xenografts are transduced with the pHR-SIN-DsRed lentiviral construct coding for constitutive expression of the Ds-Red red-fluorescent protein. This system allows for specific analysis of TMZ sensitivity ex vivo following tumor recurrence. The transduced cells are used to establish intracranial tumors in 40 mice, and mice are subsequently randomized into 4 treatment groups: A) placebo, B) 1 cycle of TMZ (66 mg/kg/day on days 1-5 of a 28 day cycle), C) 2 cycles of TMZ, and D) 3 cycles of TMZ. All mice are followed until reaching a moribund state. After euthanasia, tumor cells are recovered for evaluation of intrinsic TMZ sensitivity from 3 randomly selected and pre-determined mice from each treatment group, while the tumor/brains from the other 7 mice in each group are cross-sectioned, embedded in OCT and frozen for additional studies. Survival data for each treatment group are compared using a Cox proportional hazards model with the number of treatment cycles as a factor variable (i.e. variables with levels 0, 1, 2, and 3), with no cycles being the reference group. Point estimates and 95% confidence intervals for the hazard ratio of each level are generated. These confidence intervals are compared to determine whether there is a cycle duration effect. The correlation with the rapidity of TMZ resistance and the initial sensitivity is evaluated descriptively. The greatest number of cycles that appears to produce an additional survival benefit (determined by comparing the appropriate confidence intervals) is plotted versus the TMZ sensitivity of the tumor line. A correlation coefficient is estimated.

Tumor cells are isolated from recurrent tumors in the 3 selected animals from each treatment arm. Following removal from the cranial vault, brains are placed in ice-cold buffer, cross-sectioned in 1 mm slices and then imaged with a fluorescent microscope to detect red fluorescence. Areas containing tumor are grossly dissected and mechanically and enzymatically processed to establish a single-cell suspension. A portion of the sample is expanded in selective media and cryopreserved for further studies. The remaining cell suspension is used to evaluate TMZ sensitivity ex vivo. In conjunction with studies investigating the role of elevated MGMT activity on development of TMZ resistance, the influence of the MGMT inhibitor O6-BG on TMZ sensitivity also is evaluated. Fluorescence microscopy is used to determine the fraction and cell density of the red-fluorescent tumor cells, and 5000 red-fluorescent cells per well are plated in quadruplicate in 96 well plates. Twenty-four hours later, cells are treated with graded concentrations of TMZ (0, 20, 40, 60, 80 and 100 μM)±10 μM O6BG. The effects of TMZ therapy on cell number are monitored by fluorescence with readings obtained immediately prior to treatment and at 24 hour intervals up to 7 days. After normalizing to vehicle-treated control wells, the results derived from each of the 3 correlative animals for a given treatment group are combined in order to describe intrinsic TMZ sensitivity of tumor cells derived from each of the 4 treatment arms. Differences in in vitro TMZ sensitivity are compared across the 4 treatment arms using a general linear model: cell survival is the dependent variable and the treatment arms are the explanatory variable.

The survival benefit gained with each additional TMZ cycle can decrease with increasing cycle number. The change in efficacy may reflect progressive selection for cells intrinsically resistant to TMZ, which is measured by the ex vivo survival assay. A relatively high concentration of O6BG (10 μM) was selected to ensure that MGMT activity would be completely inhibited, and at this concentration, the TMZ resistance of MGMT-overexpressing xenograft lines is completely overcome in the xenograft model without significant toxicity from O6BG itself. An observed increase in TMZ resistance due to enhanced MGMT expression can be reversed in those samples co-treated with O6BG.

Although the tumor cell suspension used in this assay contains non-tumor cells derived from the tumor stroma and associated normal brain, by measuring changes in red fluorescence, which are only expressed by tumor cells, the effects of TMZ on tumor cell number are selectively evaluated. In other studies, expression of DsRed introduced with the lentiviral vector was stably maintained for several generations in flank tumor xenografts. The lentiviral construct also can be engineered to express a puromycin-resistance cassette and short-term puromycin selection can be used to kill contaminating murine cells prior to performing a TMZ sensitivity assay using a methylene blue survival assay. Similarly, the method for deriving short-term cell cultures from flank tumors can be adapted for use with intracranial tumors, and cells derived from these short-term cultures can be used in a methylene blue survival assay.

Tumor lines that do not develop TMZ resistance following 4 cycles of TMZ therapy are tested in a repeat study evaluating even more prolonged TMZ therapy by repeating the experiment with additional treatment arms receiving up to 8 cycles of TMZ. A lower dose intensity of TMZ with each cycle is used in follow-up experiments for more sensitive xenograft lines that are cured with multiple cycles of TMZ. Tumor lines that do not develop resistance despite extended TMZ therapy are useful to further dissect mechanisms of TMZ resistance development.

Investigating the role of elevated MGMT activity on the development of TMZ resistance: The development of TMZ resistance in the GBM12 xenograft line is associated with changes in MGMT promoter methylation detected by methylation sequencing, and these changes in MGMT methylation may be mechanistically related to the development of TMZ resistance. Heterogeneity has been reported in MGMT promoter methylation in tumor cell lines. Development of TMZ resistance during TMZ therapy may reflect a progressive enrichment of tumor cells with MGMT methylation patterns that promote high level expression of MGMT. Treatment with the specific MGMT inhibitor O6BG may reverse any observed increase in TMZ resistance in recurrent tumors. It is evaluated whether the emergence of TMZ resistance correlates with changes in MGMT mRNA and polypeptide expression and MGMT promoter methylation.

For each xenograft line tested in the previous section, tumor sections from 6 mice from each treatment group are analyzed for MGMT polypeptide and mRNA expression and MGMT promoter methylation sequencing. An MGMT IHC assay has been developed, and a similar technique is used to evaluate differences in MGMT expression levels in recurrent tumors derived from animals treated with placebo or 1 or more cycles of TMZ. As a modification of the existing assay, a FITC-labeled secondary antibody is used so that green fluorescence intensity can be used as a measure of MGMT expression (Belanich et al., Oncology Research, 6:129-137 (1994)). Since the tumor cells express DsRed, a dual-color analysis is used to ensure that only tumor cells are evaluated for expression of MGMT. For each section, 5 high-powered fields are captured and analyzed using the KS400 image analysis software. Mean fluorescence levels across the 6 tumor samples are used to describe the level of MGMT expression for each treatment group. The fluorescence intensity across the different treatment groups is compared using a general linear model with the fluorescence level as the outcome variable and treatment group as the explanatory variable. In addition to the immunofluorescence technique, tumor samples are routinely isolated from frozen brain sections, and a similar approach can be used to evaluate MGMT expression by Western blotting or MGMT activity in a O6MG repair assay.

Red fluorescence within the cryosection is used to identify areas of dense tumor and this tumor tissue is scraped from the slide and subsequently processed for mRNA quantitation and MGMT promoter methylation analysis. MGMT mRNA expression levels are quantitated using the qRT-PCR technique described above. Changes in MGMT methylation patterns are evaluated in tumor samples from each treatment arm using direct bisulfite sequencing as described above. In addition to direct sequencing, selected tumor samples are used for deep-amplicon sequencing described herein.

Changes in MGMT polypeptide expression, MGMT mRNA expression and MGMT methylation are correlated with progressive development of resistance. Changes in MGMT methylation at specific CpG sites and changes in specific CpG patterns are analyzed by methods analogous to the linear mixed models described herein. Correlations between changes in MGMT expression and changes in methylation are defined. Changes in MGMT expression or MGMT methylation are correlated with resistance development using linear regression. Increased expression of MGMT polypeptide and mRNA expression may be associated with the emergence of TMZ resistance in those tumor lines identified as being sensitized to TMZ by the MGMT inhibitor O6BG. Within a given xenograft line, the greatest changes in MGMT methylation patterns or MGMT expression levels between treatment arms may correlate with the greatest change in treatment efficacy. The CpG methylation patterns that emerge in association with TMZ resistance may be similar to those defined as being associated with intrinsic TMZ resistance in patient tumor samples.

Evaluating whether defects in MMR contribute to TMZ resistance: Tumors in which TMZ resistance is unaffected by O6BG are analyzed for possible defects in MMR. Base-base mispairing, which occurs with persistent O6MG lesions following TMZ therapy, are initially recognized by the MSH2/MSH6 heterodimer and subsequently processed in conjunction with MLH1 and PMS2. Coding sequences of MSH2, MSH6 and MLH1 are examined in recurrent TMZ-resistant tumors by direct sequencing, and changes in expression of these polypeptides are evaluated by immunohistochemistry. If mutations or silencing of MMR repair polypeptides is detected, then the functionality of the MMR process s assessed in recurrent tumors through an evaluation of microsatellite instability.

Expression levels of MMR genes are assessed by immunohistochemistry with antibodies specific for MSH2 (Clone FE11, Oncogene Science), MSH6 (clone 44, Transduction Laboratories), and MLH1 (clone G168-728, PharMingen) in tumor sections derived from placebo and TMZ-treated mice. For analysis of gene mutations, tumor is scrapped from cryosections and DNA is extracted. Individual exons are amplified by PCR and then subjected to sequencing as described elsewhere (Cunningham et al., American Journal of Human Genetics, 69:780-790 (2001) [erratum appears in Am J Hum Genet 2001 November; 69(5):1160]). If MMR polypeptide expression is suppressed or sequence mutations in the MMR genes are detected, then microsatellite instability is evaluated in the TMZ-treated tumors using 8 established microsatellite markers as described elsewhere (Thibodeau et al., Cancer Research, 56:4836-4840 (1996); Thibodeau et al., Cancer Research, 1713-1718 (1998), Cunningham et al., Cancer Research, 58:3455-3460 (1998)). In this technique, individual microsatellite regions are amplified by PCR from tumor DNA, and the PCR products are resolved by gel electrophoresis. Defects in MMR are associated with greater variability in the size of the PCR amplification products as compared to controls. In this case, the placebo-treated tumors serve as a control, and the TMZ-treated tumors are assessed for MSI.

If resistance in a specific tumor line cannot be accounted for by either changes in MGMT expression/methylation or defects in MMR, then other potential mechanisms, such as resistance to apoptosis induction or overexpression of components within the BER pathway are explored. In addition, gene expression profiling of recurrent tumor samples from placebo and TMZ-treated tumors is performed to identify other mechanisms of TMZ resistance.

Evaluating whether TMZ resistance develops by selection of pre-existing clones: Development of TMZ resistance during therapy is a major problem that limits the efficacy TMZ in a significant proportion of patients. Irrespective of promoter methylation status, disease progression during the first 6 months of TMZ therapy occurs in over 30% of patients with newly diagnosed GBM (Hegi et al., N Engl J Med, 352:997-1003 (2005)). Moreover, extended follow-up data suggest that despite promising 2 year survival rates, the vast majority of patients with MGMT promoter methylation ultimately die of recurrent GBM. Based on the data demonstrating altered MGMT methylation patterns associated with TMZ resistance development in GBM12 (FIG. 8) and observations of intratumoral heterogeneity of MGMT promoter methylation, at least a subset of GBMs may develop resistance to TMZ through selection of pre-existing tumor clones with MGMT promoter methylation patterns that confer significant resistance. This would suggest that the duration of clinical benefit from TMZ therapy may, in part, be related to the relative abundance of these sensitive vs. resistant tumor clones at the time of treatment initiation, and that tumor progression occurs when the resistant tumor clones repopulate the tumor. Detection of these TMZ-resistant clones in tumor samples prior to treatment potentially could be used to predict the rapidity of TMZ resistance development and to identify the likely mechanism of TMZ resistance. Such a predictive assay can provide individualized therapy for GBM patients in order to maximize tumor control.

The concept of detecting TMZ resistant tumor clones in previously untreated tumor samples is tested using the Roche/454 Genome Sequencer 20 pyrosequencing platform. The 454 technology relies on the hybridization of individual PCR-amplified DNA molecules onto micron-scale beads and then emulsion-PCR-based amplification of sequences on individual beads. The DNA-containing beads then are deposited onto an array in which sequencing by synthesis is monitored by emission of light catalyzed by release of pyrophosphate during addition of nucleotide bases. Because a single array allows for simultaneous sequencing of up to 420,000 DNA-containing beads, this method can be used to identify relatively rare sequences within a population of cells. This technique has been applied in studies to identify rare clones (Sogin et al., Proceedings of the National Academy of Sciences of the United States of America, 103:12115-12120 (2006); Margulies et al., Nature, 437:376-380 (2005) [erratum appears in Nature. 2006 May 4; 441(7089):120 Note: Ho, Chun He [corrected to Ho, Chun Heen]]; Thomas et al., 12: 852-855 (2006)), and to evaluate heterogeneity of DNA methylation patterns following bisulfite modification. As described herein, it is determined whether TMZ resistance development is associated with changes in MGMT promoter methylation or mutations within MMR genes. When resistance develops from pre-existing clones, then using the same tumor samples used to evaluate development of TMZ resistance to cyclical TMZ therapy, can allow detection of the initial resistant clone (or clones) in placebo-treated tumors and detection of progressive enrichment of these resistant clone(s) with successive cycles of TMZ therapy.

As described herein, the rapidity of TMZ resistance development in multiple xenograft lines is defined, and it is determined for each tumor line whether TMZ resistance emerges due to changes in MGMT promoter methylation or due to mutations or silencing of MMR genes. From these studies, 3 tumor lines displaying early, intermediate and late development of TMZ resistance are selected for analysis. The techniques and analysis for MGMT promoter methylation sequencing are described below, and similar techniques are applied to detect specific mutations within MMR genes. In conjunction with the analyses performed to investigate the role of elevated MGMT activity on the development of TMZ resistance, isolated genomic tumor DNA is purified and modified by treatment with bisulfite. The bisulfite-modified DNA is used as a template for PCR amplification of the regions of interest. Specific CpG sites that are changing with successive cycles of TMZ are identified, and these sites are encompassed by the PCR amplification strategy. Pyrosequencing provides robust sequence reads of 250 base-pairs. Any region greater than this length is evaluated with overlapping, or tiling, PCR amplicons. For example, sequencing of the entire CpG island is performed using 5 overlapping amplicons to provide coverage across the 850 by span.

In regions of less dense CpG methylation, PCR primers are designed to anneal between adjacent CpG sites so as to anneal to and amplify equally templates derived from methylated and non-methylated DNA. In regions of dense CpG sites, degenerate primers are used to recognize equally both methylated and non-methylated sequences following bisulfite treatment. To avoid amplification of murine MGMT sequences, PCR primers are designed to selectively amplify only human MGMT. The potential for primer pairs to amplify extraneous sequences within the human and mouse genome is evaluated by virtual PCR using the VPCR 2.0 web-based program. Each primer pair is tested on short-term human cell cultures derived from xenograft lines and on isolated murine genomic DNA to demonstrate selective amplification from human sequence. If the amplification strategy requires the use of degenerate primers, then the efficiency of amplification from both methylated (universally methylated DNA, Chemicon) and unmethylated templates (normal human brain DNA) is tested to demonstrate non-biased amplification of sequence regardless of methylation status. Following validation of each PCR amplicon amplification strategy, the bisulfite-treated DNA from 4 tumor samples analyzed as described above from each treatment arm are pooled and used as template for generation of the amplicon library. Each PCR amplicon is generated in a separate reaction, and PCR products are gel purified and then quantitated by spectrophotometry. If multiple tiling amplicons are used, then equimolar amounts of each amplicon for a given template are pooled. For each xenograft line, 4 distinct PCR amplicon libraries are generated that correlate with the 4 treatment arms described above (placebo, and TMZ therapy×1, 2 or 3 cycles). This PCR-amplicon library is analyzed on the 454 instrument.

Amplicon resequencing is performed in a massively parallel picoliter reaction with DNA molecules bound to micron-scale beads. The PCR amplicon library is annealed as single stranded molecules to the sequencing beads at a ratio of 0.5 DNA molecules per bead to favor the binding of a single DNA molecule to each bead. Binding specificity to beads by the PCR amplicons is conferred by a specific adapter sequence that is incorporated into the PCR primers used in the generation of the amplicon library. DNA-bound beads are encapsulated into individual micelles and subjected to emulsion PCR amplification using the GS emPCR Kit II and GS emPCR Kit III. This reaction yields beads with several million copies of the original sequence bound to each bead. The emulsion is then broken and beads are deposited onto a PicoTiterPlate device for sequencing. A 4-sample gasket is used on the PicoTiterPlate device to allow separate analysis of each of the 4 treatment arms for a given xenograft line in a single experiment.

Sequence data generated are initially analyzed using the Genome Sequencer Amplicons Variant Analysis (AVA) software from Roche/454. Prior to analysis, low-quality sequence reads are removed on the basis of the following criteria: 1) sequences that do not perfectly match the PCR primer at the beginning of the read, 2) sequence reads of <150 bp, and 3) sequences with more than 2 ambiguous base-calls. The 4 sample gasket used in the experiments provides up to 70,000 raw sequence reads per sample, and based on a study using similarly stringent sequence quality criteria (Sogin et al., Proceedings of the National Academy of Sciences of the United States of America, 103:12115-12120 (2006)), about 52,500 quality reads can be obtained for the analysis. The AVA software is used to identify the frequency of C to T conversions at each CpG site following bisulfite modification, and this provides a ratio of methylated vs. non-methylated CpGs at that site within an amplicon library. Changes in methylation status at individual sites are correlated with changes in TMZ sensitivity.

The deep methylation analysis enables an evaluation of the emergence of specific pattern(s) of CpG methylation that are associated with TMZ resistance. Specific methylation patterns within the CpG island of MGMT may be associated with robust expression of MGMT and may confer a TMZ-resistance phenotype to cells. TMZ therapy may result in the emergence of TMZ resistance through selective enrichment of a tumor cell population for those cells containing these specific methylation patterns. When this is the case, then these ‘resistant’ MGMT methylation patterns that populate the TMZ-resistant tumors can be detected at a much lower incidence in the TMZ-naïve tumors, and a progressive enrichment for these TMZ-resistant patterns can be observed with successive cycles of TMZ therapy. Over 50,000 sequence reads are performed for each treatment arm, and with a maximum of 5 amplicons being sequenced in a given analysis, this can provide a minimum sequencing depth of 10,000 reads per amplicon. To ensure that any one methylation pattern represents an authentic sequence, a minimum of 10 identical reads are required to define a specific methylation pattern. This provides a minimum sequencing power to detect rare methylation sequences at an incidence of 0.1%.

If pre-existing TMZ-resistant clones are not detected in the analysis, then TMZ resistance may be emerging due to the formation of de novo mutations or alterations in methylation. To test this possibility, clonal populations are isolated from short-term explant cultures, and the development of TMZ resistance is studied in multiple parallel clonal cultures. If de novo mutations or methylation changes account for the development of TMZ resistance, then resistance may evolve in each clonal population independently and the mutations or methylation changes may be unique for each clone. In contrast, if only a small proportion of clones develop resistance and the molecular alterations are identical across the clones, then this would suggest the emergence of resistance from some pre-existing population of clones in the original explant culture.

In addition to methylation patterns, the sequencing data provide an opportunity to examine the incidence of single nucleotide polymorphisms within the promoter that also may be associated with changes in MGMT expression and TMZ resistance.

These experiments indicate whether resistance to TMZ results from emergence of tumor cells with a ‘resistant’ MGMT promoter methylation pattern or with defects in MMR. Using deep amplicon sequencing, it is determined whether pre-existing tumor clones can be identified in tumors prior to TMZ exposure. This concept also is tested in paired patient tumor samples obtained at the time of initial diagnosis and at the time of tumor recurrence following TMZ therapy. Paired resection specimens from before and after TMZ therapy have been collected from patients. The same method of detailed deep amplicon sequence analysis is used to analyze the patient samples. The method of deep amplicon sequencing also can be applied to tumor samples from newly diagnosed patients as a predictive assay for TMZ response. Tumors with a higher proportion of TMZ resistant clones may portend an early failure on TMZ therapy, and a priori identification of the specific mechanism of TMZ resistance emergence can allow tailoring of treatment regimens to target these resistant clones during initial therapy.

### Example 3

### Correlating TMZ Responsiveness with MGMT Promoter Methylation Status and MGMT Polypeptide Expression

### Methods and Materials

Xenograft information: Thirteen serially passaged xenografts were derived from individual patients. Molecular alterations and histopathology for 11 xenografts are described elsewhere (Sarkaria et al., Mol Cancer Ther, 6:1167-1174 (2007); Sarkaria et al., Clin Cancer Res, 12:2264-2271 (2006)). Two additional xenografts, GS22 and GBM26, diagnosed as gliosarcoma and glioblastoma, respectively, were also studied.

Orthotopic model: Therapy evaluations were conducted using an orthotopic tumor model as described elsewhere (Sarkaria et al., Clin Cancer Res, 12:2264-2271 (2006), Giannini et al., Neuro-Oncology, 7:164-176, (2005)). Athymic nude mice (NCI, Frederick, Md.) with established intracranial tumors were randomized into groups of 8 to 10 mice each and treatment was initiated 2 weeks before mice were expected to become moribund. TMZ was purchased from the Mayo Clinic Pharmacy, suspended in Ora-plus (Paddock Laboratories, Minneapolis. MN), and administered by oral gavage at 66 mg/kg for 5 days. Mice were observed daily and euthanized when moribund.

Western blotting: Flank tumor specimens were processed for Western blotting as described elsewhere (Sarkaria et al., Mol Cancer Ther, 6:1167-1174 (2007)). Primary antibodies used in this study were MGMT (R&D Biosystems, Minneapolis, Minn.) and β-actin (Sigma, St. Louis, Mo.) antibodies, and secondary antibodies used were horseradish peroxidase-conjugated rabbit anti-goat and goat anti-mouse (Pierce, Rockford, Ill.) antibodies. Blots were developed with Super Signal Chemiluminescence reagent (Pierce).

MGMT Promoter Methylation Assay: DNA was extracted from flank xenograft samples using the Gentra DNA extraction kit (Puregene, Minneapolis, Minn.). Isolated tumor DNA was bisulfite-treated using the Genome Bisulfite Modification kit (Chemicon, Temecula, Calif.). The modified DNA was amplified using primers specific for either methylated or unmethylated MGMT promoter sequences as described elsewhere (Esteller et al., Cancer Res, 59:793-797 (1999); Esteller et al., N Engl J Med, 343:1350-1354 (2000)). PCR products were visualized on ethidium bromide-stained, 3% agarose gels.

MGMT methylation sequencing: The MGMT CpG island was amplified by nested PCR as described elsewhere using bisulfite-treated DNA (Matsukura et al., British Journal of Cancer, 88:521-529 (2003)). The final PCR product was incubated with shrimp-alkaline exonuclease (New England Biolab, Ipswich, Mass.) and submitted for DNA sequencing.

Statistical analysis: Survival distributions were estimated using the Kaplan-Meier method. The log rank test was used to compare survival across treatment groups. All GBM experiments were pooled and Classification and Regression Tree analysis (CART) was used to identify which CpG sites in the MS-PCR assay (CpG sites 78-82, 86-89) were predictive of overall survival in TMZ treated mice. The two-sample rank sum test was used to compare relative differences in survival relative to MGMT MS-PCR status or CpG89 methylation status. Spearman's correlation coefficient was used to assess the association of relative median survival and protein expression levels of MGMT relative to 13-actin.

### Results

Tumors in the GBM xenograft panel were established by implanting patient tumor samples subcutaneously in the flank of nude mice. The tumors were maintained through serial subcutaneous propagation. To evaluate relationships between MGMT and TMZ responsiveness, flank tumors from 13 xenograft lines were used to establish intracranial tumors for TMZ therapy evaluations. Corresponding portions of each flank tumor were examined for MGMT promoter methylation and MGMT polypeptide expression.

MGMT promoter methylation was evaluated by MS-PCR (FIG. 1A). Similar to the observed incidence of methylation in clinical samples, MGMT methylation was detected in 5 of 13 (38%) xenograft lines (GBM8, 12, 16, 36, GS22). No promoter methylation was found in the remaining 8 lines (GBM6, 10, 14, 26, 34, 43, 44, GS28). Consistent with gene silencing via promoter methylation, MGMT methylation was associated with low to undetectable levels of MGMT polypeptide (FIG. 1B). In contrast, significant variation in MGMT expression was evident in non-methylated tumors, which ranged from robust expression (GBM10 and 43) to undetectable (GBM14 and 34).

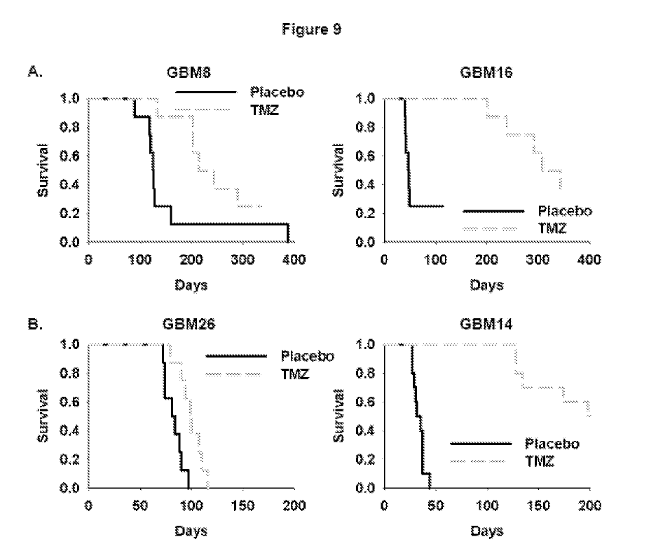

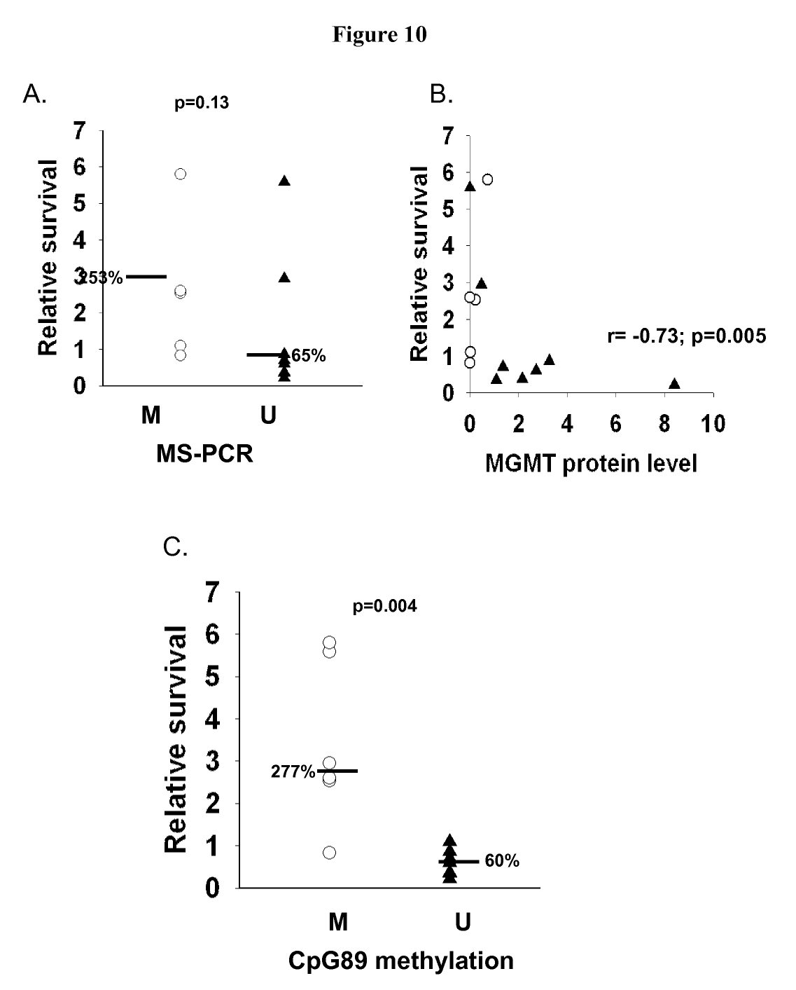

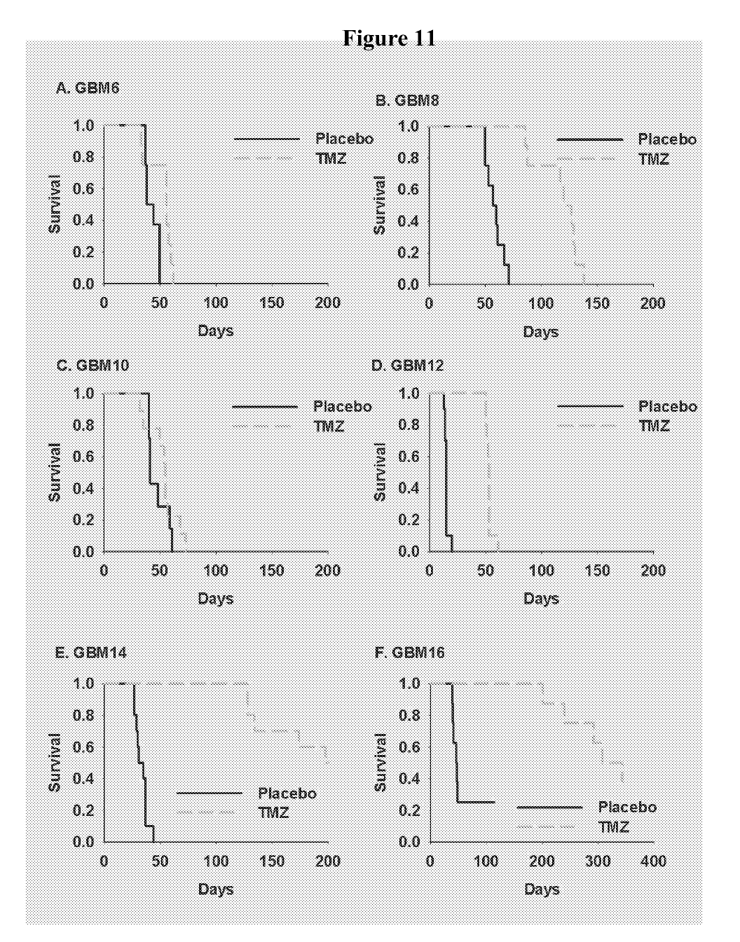

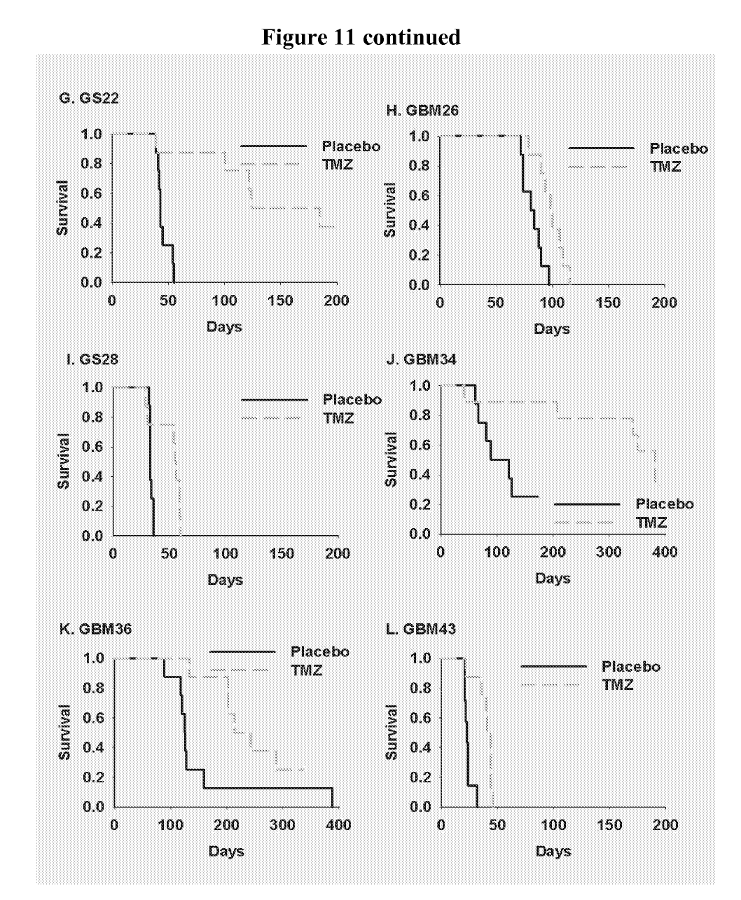

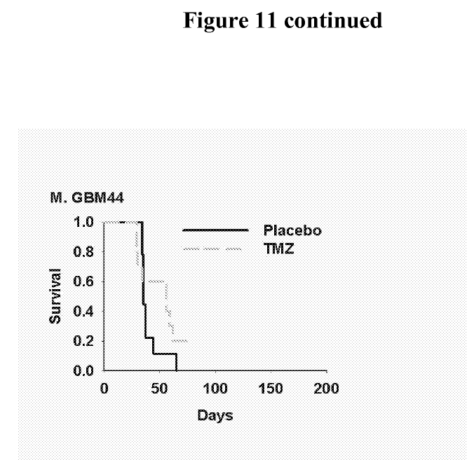

TMZ sensitivity was evaluated using an orthotopic model. Mice with established intracranial tumors were randomized to placebo or TMZ treatment for 5 days, and the mice were followed for survival (Table 3 and FIG. 11). There was a broad range of TMZ responsiveness, with treatment extending the median survival of mice from 21% (GBM26) to 579% (GBM16; FIG. 9) relative to placebo. The relationship between TMZ responsiveness and MGMT status was evaluated by comparing the distribution of survival prolongation for methylation-positive and methylation-negative lines. MGMT hypermethylation was associated with a greater prolongation in survival (FIG. 10A; median survival benefit 253%, range 82-579%), as compared to non-methylated tumors (median survival benefit 65%, range 21-558%; p=0.13), although this association was not significant. In a similar analysis, MGMT polypeptide expression levels from FIG. 1B were measured by film densitometry, and normalized values were plotted relative to survival benefit (FIG. 10B). This demonstrated an inverse relationship between MGMT polypeptide levels and TMZ resistance (r=−0.75; p=0.003), with high level MGMT expression invariably associated with TMZ resistance. For tumors with a low but detectable MGMT expression (GBM8, 12, 16, 36, GS28) there was considerable variability in survival benefit ranging from 70% to 579% prolongation in median survival. Neither low MGMT expression nor MGMT promoter methylation was entirely reliable in predicting TMZ responsiveness in this orthotopic model.

TABLE 3
Survival summary

| | Placebo | | Temozolomide | | | | | |
|---|---|---|---|---|---|---|---|---|
| | | median | | median | | Rank-sum | Methylation status | |
| | n | survival | n | survival | Survival Prolongation | p-value | MS-PCR | CpG89 |
| GBM6 | 8 | 41 | 8 | 56 | 37% | 0.104 | U | U |
| GBM8 | 8 | 59 | 8 | 124 | 110% | <0.001 | M | U |
| GBM10 | 8 | 41 | 9 | 55 | 34% | 0.370 | U | U |
| GBM12 | 10 | 15 | 10 | 53 | 253% | <0.001 | M | M |
| GBM14 | 10 | 33 | 10 | 217 | 558% | <0.001 | U | M |
| GBM16 | 8 | 48 | 8 | 326 | 579% | 0.006 | M | M |
| GS22 | 8 | 43 | 8 | 155 | 260% | 0.006 | M | M |
| GBM26 | 8 | 83 | 8 | 100 | 21% | 0.006 | U | U |
| GS28 | 8 | 33 | 8 | 56 | 70% | 0.104 | U | U |
| GBM34 | 7 | 89 | 9 | 351 | 294% | 0.008 | U | M |
| GBM36 | 8 | 126 | 8 | 229 | 82% | 0.014 | M | U |
| GBM43 | 8 | 23 | 8 | 43 | 87% | 0.002 | U | U |
| GBM44 | 9 | 35 | 10 | 56 | 60% | 0.660 | U | U |

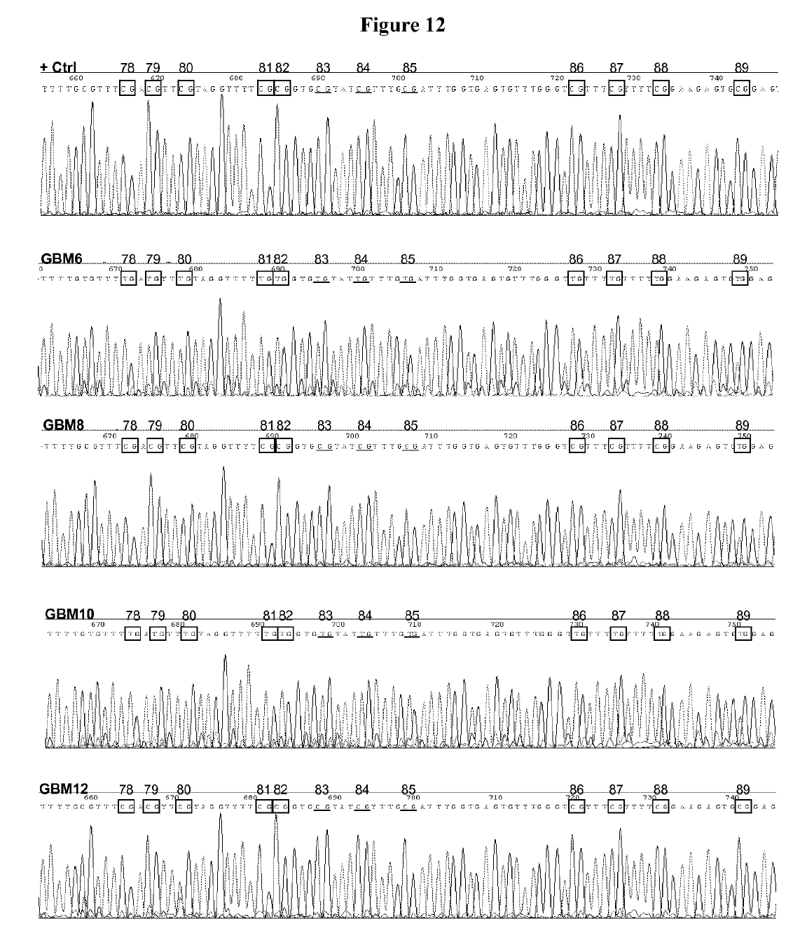

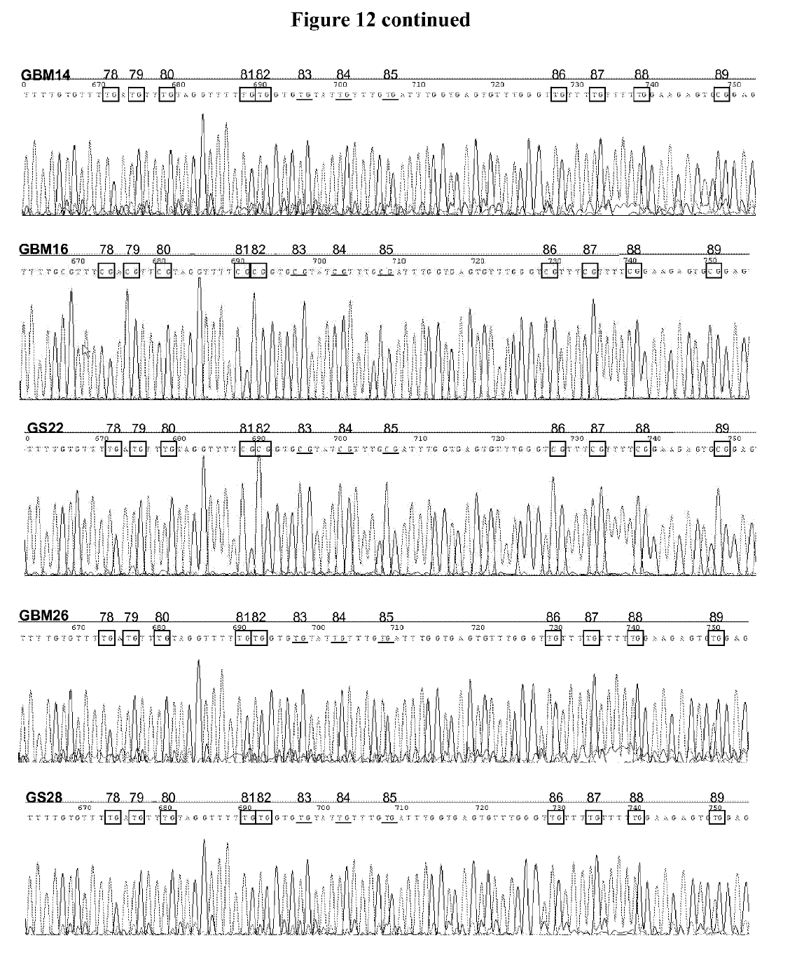

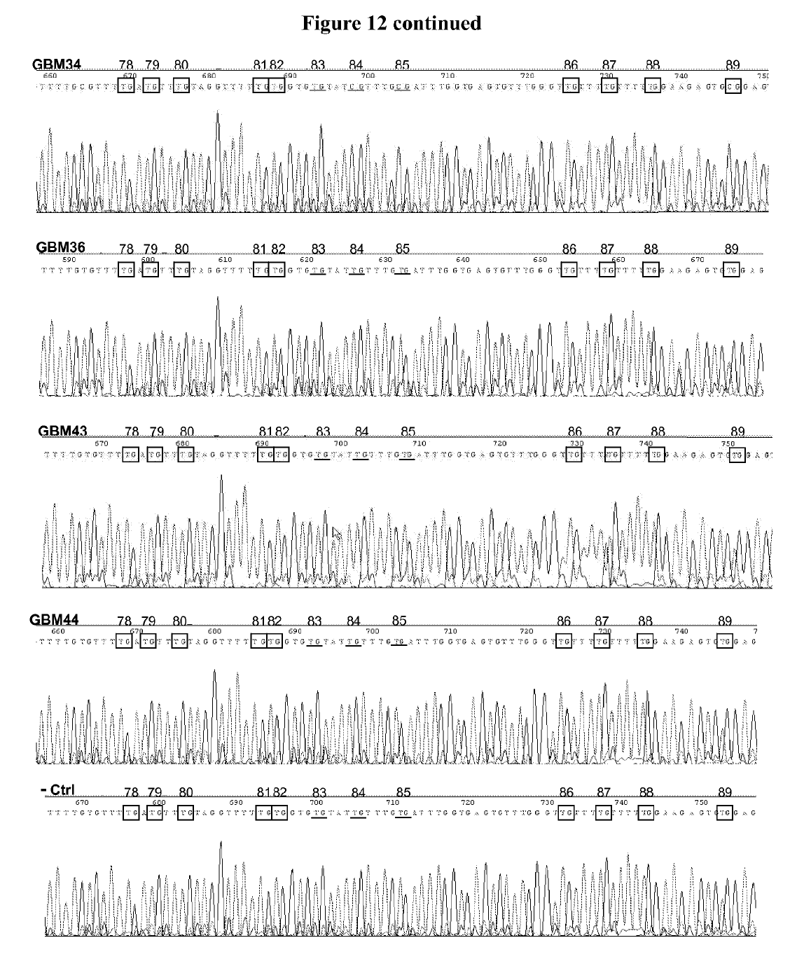

Determination of MGMT methylation status by MS-PCR relies on hybridization of primers across nine CpG sites (CpG 78-82 and CpG 86-89) and does not delineate the methylation status of individual CpG sites. To address possible heterogeneity of methylation across these sites, methylation-specific sequencing was performed (FIG. 12). For the most part, CpG sites were either wholly methylated or un-methylated, although CpG 80 in GBM12 and CpG 87 in GBM43 were hemi-methylated. The sequencing results were tabulated and compared with the MS-PCR results (FIG. 5). In GBM36, which is methylated by MS-PCR, there was discordance between the two methods, with all sites found to be unmethylated by MS-sequencing. For the other lines, there was concordance between MS-PCR and MS-sequencing results for CpG 81, 82, 86, and 88. For the other five sites, methylation status defined by MS-PCR and by sequencing was discordant in at least one line. CpG 89 methylation was the most divergent between the MS-PCR and sequencing assessment, and methylation at CpG 89 was highly correlated with TMZ sensitivity (FIG. 10C). The median survival benefit was 277% (range 82-579%) for tumors with CpG89 methylation, as compared to 60% (range 21-110%) without CpG89 methylation (p=0.004).

The results of these studies indicated that xenografts with MGMT hypermethylation were generally more sensitive to TMZ than xenografts without hypermethylation (median survival benefit 253% vs. 65%), although this association did not reach statistical significance (p=0.13) with the number of lines assessed (n=13). Similar to clinical observations (2, 4, 15), a subset of those xenografts with MGMT hypermethylation were relatively resistant to TMZ therapy (GBM8 and 36), and conversely, a subset of xenografts lacking MGMT hypermethylation were highly sensitive to TMZ therapy (GBM14 and 34). MGMT methylated tumors expressed low levels of MGMT polypeptide (FIG. 1). A subset of tumors lacking MGMT methylation also had low MGMT polypeptide levels (GBM14, GS28 and GBM34) similar to those seen in some MGMT methylated tumors (GBM12, GBM16 and GBM36). In the subset of low MGMT-expressing tumors, there was a broad range in TMZ responsiveness with prolongation in survival ranging from 70% to 579%.

The methylation status of one or more CpG sites was discordant with the MS-PCR results in 7 of 13 xenograft lines, and the sequencing-assessed status of one of these sites, CpG 89, was highly correlated with TMZ sensitivity (FIG. 10C; p=0.004). In relation to specific xenograft lines, CpG89 methylation in the MS-PCR unmethylated GBM14 and GBM34 lines was associated with significant TMZ sensitivity, and lack of methylation at this site was associated with relative TMZ resistance in the MS-PCR methylated GBM8 and GBM36 tumor lines. In the relatively resistant GBM36 tumor, MS-sequencing revealed lack of methylation at all CpG sites, while MS-PCR demonstrated amplification of both unmethylated and methylated products. These results likely reflect heterogeneous methylation of the MGMT promoter with MS-PCR detecting a small fraction of cells with MGMT promoter methylation (18, 19). These data suggest that evaluation of methylation at individual CpG sites may provide a more robust predictor of TMZ responsiveness than the MGMT MS-PCR assay.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining whether or not a mammal having cancer is responsive to treatment with chemotherapy, said method comprising determining the methylation status of CpG 89 in an MGMT nucleic acid in a tumor of said mammal, wherein methylation at CpG 89 indicates that said mammal is responsive to treatment with chemotherapy, and lack of methylation at CpG 89 indicates that said mammal is not responsive to treatment with chemotherapy.

2. The method of claim 1, wherein said mammal is human.

3. The method of claim 1, wherein said cancer is brain cancer.

4. The method of claim 1, wherein said chemotherapy is temozolomide.

5. The method of claim 1, wherein said brain cancer is glioblastoma multiforme.

6. The method of claim 1, wherein said methylation status of CpG 89 is determined using methylation sequencing.

7. The method of claim 1, wherein a mammal that is responsive to treatment with chemotherapy and is treated with chemotherapy is likely to survive longer than a mammal that is not responsive to treatment with chemotherapy and is treated with chemotherapy.


SEQUENCE LISTING (33 sequences)

SEQ ID NO: 1 - DNA, 817 bp, Homo sapiens
cggccccctgacagggtctctgctggtctgggggtccctgactaggggagcggcaccaggaggggagagactcgcgctccgggctcagcgtagccgccccgagcaggaccgggattctcactaagcgggcgccgtcctacgacccccgcgcgctttcaggaccactcgggcacgtggcaggtcgcttgcacgcccgcggactatccctgtgacaggaaaaggtacgggccatttggcaaactaaggcacagagcctcaggcggaagctgggaaggcgccgcccggcttgtaccggccgaagggccatccgggtcaggcgcacagggcagcggcgctgccggaggaccagggccggcgtgccggcgtccagcgaggatgcgcagactgcctcaggcccggcgccgccgcacagggcatgcgccgacccggtcgggcgggaacaccccgcccctcccgggctccgccccagctccgcccccgcgcgccccggccccgcccccgcgcgctctcttgcttttctcaggtcctcggctccgccccgctctagaccccgccccacgccgccatccccgtgcccctcggccccgcccccgcgccccggatatgctgggacagcccgcgcccctagaacgctttgcgtcccgacgcccgcaggtcctcgcggtgcgcaccgtttgcgacttggtgagtgtctgggtcgcctcgctcccggaagagtgcggagctctccctcgggacggtggcagcctcgagtggtcctgcaggcgccctcacttcgccgtcgggtgtggggccgccctgacccccacccatcccg

SEQ ID NO: 2 - DNA, 29 bp, Homo sapiens
tttgtgttttgatgtttgtaggtttttgt

SEQ ID NO: 3 - DNA, 28 bp, Homo sapiens
aactccacactcttccaaaaacaaaaca

SEQ ID NO: 4 - DNA, 23 bp, Homo sapiens
tttcgacgttcgtaggttttcgc

SEQ ID NO: 5 - DNA, 22 bp, Homo sapiens
gcactcttccgaaaacgaaacg

SEQ ID NO: 6 - DNA, 23 bp, Artificial Sequence
  synthetically generated oligonucleotide
ttgganggtatngttattatagg

SEQ ID NO: 7 - DNA, 26 bp, Homo sapiens
cctaaaactctataccttaattaacc

SEQ ID NO: 8 - DNA, 25 bp, Homo sapiens
ttattataggttttggaggttgttt

SEQ ID NO: 9 - DNA, 26 bp, Homo sapiens
cctaaaactctataccttaattaacc

SEQ ID NO: 10 - DNA, 26 bp, Homo sapiens
ggtaaattaaggtatagagttttagg

SEQ ID NO: 11 - DNA, 24 bp, Artificial Sequence
  synthetically generated oligonucleotide
aataaataaaaatcaaaacnaccc

SEQ ID NO: 12 - DNA, 26 bp, Homo sapiens
ggtaaattaaggtatagagttttagg

SEQ ID NO: 13 - DNA, 20 bp, Artificial Sequence
  synthetically generated oligonucleotide
aaaaatcaaaacnacccccc

SEQ ID NO: 14 - DNA, 20 bp, Homo sapiens
cctggctgaatgcctatttc

SEQ ID NO: 15 - DNA, 21 bp, Homo sapiens
gatgaggatggggacaggatt

SEQ ID NO: 16 - DNA, 20 bp, Homo sapiens
ttctggcctggagggcatcc

SEQ ID NO: 17 - DNA, 20 bp, Homo sapiens
atcttcaaacctccatgatg

SEQ ID NO: 18 - DNA, 92 bp, Homo sapiens
ctttgcgtcccgacgcccgcaggtcctcgcggtgcgcaccgtttgcgacttggtgagtgtctgggtcgcctcgctcccggaagagtgcggag

SEQ ID NO: 19 - DNA, 92 bp, Homo sapiens
ttttgcgtttcgacgttcgtaggttttcgcggtgcgtatcgtttgcgatttggtgagtgtttgggtcgtttcgttttcggaagagtgcggag

SEQ ID NO: 20 - DNA, 92 bp, Homo sapiens
ttttgtgttttgatgtttgtaggtttttgtggtgtgtattgtttgtgatttggtgagtgtttgggttgttttgtttttggaagagtgtggag

SEQ ID NO: 21 - DNA, 92 bp, Homo sapiens
ttttgcgtttcgacgttcgtaggttttcgcggtgcgtatcgtttgcgatttggtgagtgtttgggtcgtttcgttttcggaagagtgtggag

SEQ ID NO: 22 - DNA, 92 bp, Homo sapiens; identical to SEQ ID NO: 20

SEQ ID NO: 23 - DNA, 92 bp, Homo sapiens; identical to SEQ ID NO: 19

SEQ ID NO: 24 - DNA, 92 bp, Homo sapiens
ttttgtgttttgatgtttgtaggtttttgtggtgtgtattgtttgtgatttggtgagtgtttgggttgttttgtttttggaagagtgcggag

SEQ ID NO: 25 - DNA, 92 bp, Homo sapiens; identical to SEQ ID NO: 19

SEQ ID NO: 26 - DNA, 92 bp, Homo sapiens
ttttgtgttttgatgtttgtaggttttcgcggtgcgtatcgtttgcgatttggtgagtgtttgggtcgtttcgttttcggaagagtgcggag

SEQ ID NO: 27 - DNA, 92 bp, Homo sapiens; identical to SEQ ID NO: 20

SEQ ID NO: 28 - DNA, 92 bp, Homo sapiens; identical to SEQ ID NO: 20

SEQ ID NO: 29 - DNA, 92 bp, Homo sapiens
ttttgcgttttgatgtttgtaggtttttgtggtgtgtatcgtttgcgatttggtgagtgtttgggttgttttgtttttggaagagtgcggag

SEQ ID NO: 30 - DNA, 92 bp, Homo sapiens; identical to SEQ ID NO: 20

SEQ ID NO: 31 - DNA, 92 bp, Homo sapiens; identical to SEQ ID NO: 20

SEQ ID NO: 32 - DNA, 92 bp, Homo sapiens; identical to SEQ ID NO: 20

SEQ ID NO: 33 - DNA, 91 bp, Homo sapiens
ttttgtgttttgatgtttgtaggtttttgtggtgtgtattgtttgtgatttggtgagtgtttgggttgttttgtttttggaagagtgtgag